(19) United States
(12) Patent Application Publication
(10) Pub. No.: US 2022/0296535 A1
(43) Pub. Date: Sep. 22, 2022

(54) Antibacterial Dosage Regime Using Cannabinoids

(71) Applicant: Botanix Pharmaceuticals Limited, North Perth (AU)

(72) Inventors: Matthew Callahan, Philadelphia, PA (US); Michael Thurn, New South Wales (AU)

(21) Appl. No.: 17/617,453

(22) PCT Filed: Jun. 17, 2020
(86) PCT No.: PCT/AU2020/050607

§ 371 Date: Dec. 8, 2021

(30) Foreign Application Priority Data
Jun. 18, 2019 (AU) 2019 902124

(51) Int. Cl. A61K 31/05; A61P 31/04; A61K 47/10; A61K 47/34; A61K 9/00

(52) U.S. Cl. CPC A61K 31/05; A61P 31/04; A61K 47/10; A61K 47/34; A61K 9/0073; A61K 9/0048; A61K 9/0014

(57) ABSTRACT

The present invention provides methods, compositions and uses for treating topical bacterial infections comprising the administration of cannabinoids. In particular, the present invention provides methods, uses and compositions for treating bacterial infections, the compositions comprising cannabidiol or other cannabinoids, in a dose ranging from about 25 mg to about 500 mg of the cannabinoid compounds.

### TECHNICAL FIELD

A dosage regimen for the treatment or prevention of bacterial infections, comprising the use of a cannabinoid.

### BACKGROUND ART

Compounds with antimicrobial properties have attracted great interest in recent times as a result of an increase in the prevalence of infections caused by bacteria, resulting in serious or fatal diseases. Furthermore, the regular use of broad-spectrum antibiotics has led to the increased occurrence of bacterial strains that are resistant to some antimicrobial compositions, such as methicillin-resistant Staphylococcus aureus (MSRA).

Novel antimicrobial compounds and new compositions have the potential to be highly effective against these types of antibiotic-resistant bacteria. The pathogens, having not previously been exposed to the antimicrobial composition, may have little to no resistance to the treatment.

There is no indication that bacterial resistance to antibiotics will stop and for this reason new antibiotics and new treatment options are necessary to achieve a desirable treatment outcome in human and non-human subjects.

Many microbes form highly organised structures called biofilms in which they are protected from immune cells and antibiotic killing via several mechanisms. These mechanisms include reduced antibiotic penetration, low metabolic activity, physiological adaptation, antibiotic-degrading enzymes, and selection for genetically resistant variants (Stewart & Costerton Lancet. 2001 358(9276):135-138).

There is a need to provide new dosing regimens for the treatment of infections by bacteria, particularly bacteria resistant to the current antibiotic compounds available. This invention seeks to provide such alternative dosing regimens.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

According to one aspect of the invention, there is provided a topical dosing regimen for the treatment or prevention of an infection in a subject by a bacterium, said regimen comprising the steps of:
administering between 25 mg and 500 mg of a cannabinoid to the subject.

Preferably, the topical dosing regimen is a dosing regimen applied to the skin and mucosal surfaces (e.g. oral membranes, vaginal membranes, rectal membranes), ocular surfaces or nasal surfaces.

Preferably the topical dosing regimen results in the bacterial decolonisation of the skin and mucosal surface, ocular surface or nasal surface.

Preferably the dosing regime delivers a composition in the form of a gel composition or ointment composition. The ointment composition preferable comprises one or more poly (substituted or unsubstituted alkylene) glycol or a derivative thereof. The gel composition preferably comprises a volatile solvent to dissolve the cannabinoid (e.g. a siloxane and/or a low molecular weight alcohol), and a viscosity modifier to increase the viscosity.

According to the invention, there is also provided a topical dosing regimen applied to the skin and mucosal surfaces for the treatment or prevention of a topical infection of a subject by a bacterium, said regimen comprising the steps of:
administering a topical composition between 25 mg and 500 mg of a cannabinoid to the subject.

According to the invention, there is also provided an ocular dosing regimen for the treatment or prevention of an ocular infection of a subject by a bacterium, said regimen comprising the steps of:
administering a topical composition between 25 mg and 500 mg of a cannabinoid to the site of the ocular infection.

According to the invention, there is also provided a topical nasal or inhaled dosing regimen for the treatment or prevention of an infection in a subject by a bacterium, said regimen comprising the steps of:
administering by nasal delivery or inhalation between 25 mg and 500 mg of a cannabinoid to the subject.

According to another aspect of the invention, there is provided a method for the treatment or prevention of a topical infection by a bacterium in a subject in need of such treatment, said method comprising the step of:
topically administering a composition comprising between 25 mg and 500 mg of a cannabinoid to the subject.

Preferably, the method of treatment or prevention of a topical bacterial infection is a topical dosing regimen applied to the skin and mucosal surfaces (e.g. oral membranes, vaginal membranes, rectal membranes), ocular surfaces or nasal surfaces.

Preferably the method of treatment or prevention of a bacterial infection results in the bacterial decolonisation of the skin and mucosal surface, ocular surface or nasal surface.

According to the invention, there is also provided a method for the treatment or prevention of a topical infection by a bacterium in a subject in need of such treatment, said method comprising the step of:
administering to the skin and mucosal surfaces a topical composition comprising between 25 mg and 500 mg of a cannabinoid to the subject.

According to the invention, there is also provided a method for the treatment or prevention of an ocular infection by a bacterium in a subject in need of such treatment, said method comprising the step of:
administering a topical ocular composition comprising between 25 mg and 500 mg of a cannabinoid to the subject.

According to the invention, there is also provided a method for the treatment or prevention of a topical infection by a bacterium in a subject in need of such treatment, said method comprising the step of:
administering a topical nasal or inhaled composition comprising between 25mg and 500 mg of a cannabinoid to the subject by injection.

According to another aspect of the invention, there is provided the use of a topical composition comprising between 25 mg and 500 mg of a cannabinoid for the treatment or prevention of a topical bacterial infection in a subject in need of such treatment or prevention.

Preferably, the use is a dosing regimen applied to the skin and mucosal surfaces (e.g. oral membranes, vaginal membranes, rectal membranes), ocular surfaces or nasal surfaces.

According to the invention, there is also provided the use of a topical composition comprising between 25 mg and 500 mg of a cannabinoid for administration to the skin and mucosal surfaces for the treatment or prevention of a topical bacterial infection in a subject in need of such treatment or prevention.

According to the invention, there is also provided the use of a topical ocular composition comprising between 25 mg and 500 mg of a cannabinoid for the treatment or prevention of an ocular bacterial infection in a subject in need of such treatment or prevention.

According to the invention, there is also provided the use of a topical nasal or inhaled composition comprising between 25 mg and 500mg of a cannabinoid for the treatment or prevention of a bacterial infection in a subject in need of such treatment or prevention.

According to another aspect of the invention, there is provided the use of a cannabinoid for the manufacture of a topical composition for the treatment or prevention of a topical bacterial infection, wherein between 25 mg and 500 mg of the cannabinoid is administered to a subject in need of such treatment or prevention.

According to the invention, there is also provided the use of a cannabinoid for the manufacture of a topical composition for administration to the skin and mucus surfaces for the treatment or prevention of a topical bacterial infection, wherein between 25 mg and 500 mg of the cannabinoid is administered to a subject in need of such treatment or prevention.

According to the invention, there is also provided the use of a cannabinoid for the manufacture of a topical ocular composition for the treatment or prevention of an ocular bacterial infection, wherein between 25 mg and 500 mg of the cannabinoid is administered to a subject in need of such treatment or prevention.

According to the invention, there is also provided the use of a cannabinoid for the manufacture of a topical nasal or inhaled composition for the treatment or prevention of a bacterial infection, wherein between 25 mg and 500 mg of the cannabinoid is administered by a nasal or inhaled dosing regimen to a subject in need of such treatment or prevention.

According to another aspect of the invention, there is provided the manufacture of a topical composition comprising a cannabinoid for use in the treatment or prevention of a topical bacterial infection, wherein between 25 mg and 500 mg of the cannabinoid is administered to a subject in need of such treatment or prevention.

According to the invention, there is also provided the manufacture of a topical composition for administration to the skin and mucosal surfaces comprising a cannabinoid for use in the treatment or prevention of a topical bacterial infection, wherein between 25 mg and 500 mg of the cannabinoid is administered topically to a subject in need of such treatment or prevention.

According to the invention, there is also provided the manufacture of a topical ocular composition comprising a cannabinoid for use in the treatment or prevention of an ocular bacterial infection, wherein between 25 mg and 500 mg of the cannabinoid is administered to the site of the ocular infection in a subject in need of such treatment or prevention.

According to the invention, there is also provided the manufacture of a topical nasal or inhaled composition comprising a cannabinoid for use in the treatment or prevention of a bacterial infection, wherein between 25 mg and 500 mg of the cannabinoid is administered by a nasal or inhaled dosing regimen to a subject in need of such treatment or prevention.

According to another aspect of the invention, there is provided a topical composition comprising a cannabinoid for use in the treatment or prevention of a topical bacterial infection, wherein between 25 mg and 500 mg of the cannabinoid is administered topically to a subject in need of such treatment or prevention.

According to the invention, there is also provided a topical composition for administration to the skin and mucosal surfaces comprising a cannabinoid for use in the treatment or prevention of a topical bacterial infection, wherein between 25 mg and 500 mg of the cannabinoid is administered topically to the skin and mucosal surfaces of a subject in need of such treatment or prevention.

According to the invention, there is also provided a topical ocular composition comprising a cannabinoid for use in the treatment or prevention of an ocular bacterial infection, wherein between 25 mg and 2000 mg of the cannabinoid is administered to the site of the ocular infection a subject in need of such treatment or prevention.

According to the invention, there is also provided a topical nasal or inhaled composition comprising a cannabinoid for use in the treatment or prevention of a bacterial infection, wherein between 25 mg and 500 mg of the cannabinoid is administered by a nasal or inhaled dosing regimen to a subject in need of such treatment or prevention.

In a preferred form of the invention, the bacterium is a Gram-positive bacterium.

In a preferred form of the invention, the bacterium is a bacterium species of a genus selected from the list: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof.

In a preferred form of the invention, the bacterium is a biofilm-forming bacterium.

In a preferred form of the invention, the bacterium is resistant to at least one antibiotic.

Preferably the cannabinoid is cannabidiol.

Preferably the composition of the present invention is in the form of a gel composition or ointment composition. The ointment composition preferable comprises one or more poly (substituted or unsubstituted alkylene) glycol or a derivative thereof. The gel composition preferably comprises a volatile solvent to dissolve the cannabinoid (e.g. a siloxane and/or a low molecular weight alcohol), and a viscosity modifier to increase the viscosity.

### DESCRIPTION OF INVENTION

### Treatment Regime

According to one aspect of the invention, there is provided a topical dosing regimen for the treatment or prevention of an infection in a subject by a bacterium, said regimen comprising the steps of:
administering between 25 mg and 500 mg of a cannabinoid to the subject.

According to the invention, there is also provided a topical dosing regimen applied to the skin and mucosal surfaces for the treatment or prevention of a topical infection of a subject by a bacterium, said regimen comprising the steps of:
topically administering between 25 mg and 500 mg of a cannabinoid to the subject.

According to the invention, there is also provided topical ocular dosing regimen for the treatment or prevention of an ocular infection of a subject by a bacterium, said regimen comprising the steps of:
administering between 25 mg and 500 mg of a cannabinoid to the site of the ocular infection.

According to the invention, there is also provided a topical nasal or inhaled dosing regimen for the treatment or prevention of an infection in a subject by a bacterium, said regimen comprising the steps of:
administering by nasal delivery or inhalation between 25 mg and 500 mg of a cannabinoid to the subject.

According to one aspect of the invention, there is provided a topical dosing regimen for the bacterial decolonisation of the skin and mucosal surface, ocular surface or nasal surface in a subject, said regimen comprising the steps of:
administering between 25 mg and 500 mg of a cannabinoid to the subject.

Preferably, the topical dosing regimen is a dosing regimen applied to the skin and mucosal surfaces (e.g. oral membranes, vaginal membranes, rectal membranes), ocular surfaces or nasal surfaces.

Preferably the topical dosing regimen results in the bacterial decolonisation of the skin and mucosal surface, ocular surface or nasal surface.

Preferably the bacterium is a biofilm-forming bacterium. Preferably the bacterium is an antibiotic resistant bacterium. The bacterium may be both biofilm-forming and antibiotic resistant.

In a preferred form of the invention, the bacterium is a Gram-positive bacterium, preferably a bacterium species of a genus selected from the list: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof.

Preferably, the cannabinoid is chosen from the list comprising: cannabidiol, cannabinol, cannabigerol, cannabichromene, and Δ⁹-tetrahydrocannabinol. Most preferably, the cannabinoid is cannabidiol.

The compositions may contain more than one cannabinoid. For example, the composition of the present invention may contain a combination of two, three or more cannabinoids.

### Method of Treatment

According to another aspect of the invention, there is provided a method for the treatment or prevention of a topical infection by a bacterium in a subject in need of such treatment, said method comprising the step of:
topically administering a composition comprising between 25 mg and 500 mg of a cannabinoid to the subject.

According to the invention, there is also provided a method for the treatment or prevention of a topical infection by a bacterium in a subject in need of such treatment, said method comprising the step of:
administering to the skin and mucosal surfaces a topical composition comprising between 25 mg and 500 mg of a cannabinoid to the subject.

According to the invention, there is also provided a method for the treatment or prevention of an ocular infection by a bacterium in a subject in need of such treatment, said method comprising the step of:
administering a topical ocular composition comprising between 25 mg and 500 mg of a cannabinoid to the subject.

According to the invention, there is also provided a method for the treatment or prevention of a topical infection by a bacterium in a subject in need of such treatment, said method comprising the step of:
administering a nasal or inhaled composition comprising between 25 mg and 500 mg of a cannabinoid to the subject by injection.

According to another aspect of the invention, there is provided a method for the topical bacterial decolonisation of the skin and mucosal surface, ocular surface or nasal surface a subject in need of such treatment, said method comprising the step of:
topically administering a composition comprising between 25 mg and 500 mg of a cannabinoid to the subject.

Preferably, the method for the treatment or prevention of a topical infection by a bacterium is a dosing regimen applied to the skin and mucosal surfaces (e.g. oral membranes, vaginal membranes, rectal membranes), ocular surfaces or nasal surfaces.

Preferably the method of treatment or prevention of a topical bacterial infection results in the bacterial decolonisation of the skin and mucosal surface, ocular surface or nasal surface.

Preferably the bacterium is a biofilm-forming bacterium. Preferably the bacterium is an antibiotic resistant bacterium. The bacterium may be both biofilm-forming and antibiotic resistant.

In a preferred form of the invention, the bacterium is a Gram-positive bacterium, preferably a bacterium species of a genus selected from the list: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof.

Preferably, the cannabinoid is chosen from the list comprising: cannabidiol, cannabinol, cannabigerol, cannabichromene, and Δ⁹-tetrahydrocannabinol. Most preferably, the cannabinoid is cannabidiol.

The compositions for use in the method of the invention may contain more than one cannabinoid. For example, the composition of the present invention may contain a combination of two, three or more cannabinoids.

### Use

According to another aspect of the invention, there is provided the use of a topical composition comprising between 25 mg and 500 mg of a cannabinoid for the treatment or prevention of a topical bacterial infection in a subject in need of such treatment or prevention.

According to the invention, there is also provided the use of a topical composition comprising between 25 mg and 500 mg of a cannabinoid for administration to the skin and mucosal surfaces for the treatment or prevention of a topical bacterial infection in a subject in need of such treatment or prevention.

According to the invention, there is also provided the use of a topical ocular composition comprising between 25 mg and 500 mg of a cannabinoid for the treatment or prevention of an ocular bacterial infection in a subject in need of such treatment or prevention.

According to the invention, there is also provided the use of a topical nasal or inhaled composition comprising between 25 mg and 500 mg of a cannabinoid for the treatment or prevention of a bacterial infection in a subject in need of such treatment or prevention.

Preferably the use results in the bacterial decolonisation of the skin and mucosal surface, ocular surface or nasal surface.

Preferably, the use is a dosing regimen applied to the skin and mucosal surfaces (e.g. oral membranes, vaginal membranes, rectal membranes), ocular surfaces or nasal surfaces.

Preferably the bacterium is a biofilm-forming bacterium. Preferably the bacterium is an antibiotic resistant bacterium. The bacterium may be both biofilm-forming and antibiotic resistant.

In a preferred form of the invention, the bacterium is a Gram-positive bacterium, preferably a bacterium species of a genus selected from the list: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof.

Preferably, the cannabinoid is chosen from the list comprising: cannabidiol, cannabinol, cannabigerol, cannabichromene, and Δ⁹-tetrahydrocannabinol. Most preferably, the cannabinoid is cannabidiol.

According to another aspect of the invention, there is provided the use of a cannabinoid for the manufacture of a topical composition for the treatment or prevention of a topical bacterial infection, wherein between 25 mg and 500 mg of the cannabinoid is administered to a subject in need of such treatment or prevention.

According to the invention, there is also provided the use of a cannabinoid for the manufacture of a topical composition for administration to the skin and mucus surfaces for the treatment or prevention of a topical bacterial infection, wherein between 25 mg and 500 mg of the cannabinoid is administered to a subject in need of such treatment or prevention.

According to the invention, there is also provided the use of a cannabinoid for the manufacture of a topical ocular composition for the treatment or prevention of an ocular bacterial infection, wherein between 25 mg and 500 mg of the cannabinoid is administered to a subject in need of such treatment or prevention.

According to the invention, there is also provided the use of a cannabinoid for the manufacture of a topical nasal or inhaled composition for the treatment or prevention of a bacterial infection, wherein between 25 mg and 500 mg of the cannabinoid is administered by a nasal or inhaled dosing regimen to a subject in need of such treatment or prevention.

Preferably the use results in the bacterial decolonisation of the skin and mucosal surface, ocular surface or nasal surface.

Preferably, the use is a dosing regimen applied to the skin and mucosal surfaces (e.g. oral membranes, vaginal membranes, rectal membranes), ocular surfaces or nasal surfaces.

Preferably the bacterium is a biofilm-forming bacterium. Preferably the bacterium is an antibiotic resistant bacterium. The bacterium may be both biofilm-forming and antibiotic resistant.

In a preferred form of the invention, the bacterium is a Gram-positive bacterium, preferably a bacterium species of a genus selected from the list: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof.

Preferably, the cannabinoid is chosen from the list comprising: cannabidiol, cannabinol, cannabigerol, cannabichromene, and Δ⁹-tetrahydrocannabinol. Most preferably, the cannabinoid is cannabidiol.

According to another aspect of the invention, there is provided the manufacture of a topical composition comprising a cannabinoid for use in the treatment or prevention of a topical bacterial infection, wherein between 25 mg and 500 mg of the cannabinoid is administered to a subject in need of such treatment or prevention.

According to the invention, there is also provided the manufacture of a topical composition comprising a cannabinoid for administration to the skin and mucus surfaces for use in the treatment or prevention of a topical bacterial infection, wherein between 25 mg and 500 mg of the cannabinoid is administered topically to a subject in need of such treatment or prevention.

According to the invention, there is also provided the manufacture of a topical ocular composition comprising a cannabinoid for use in the treatment or prevention of an ocular bacterial infection, wherein between 25 mg and 500 mg of the cannabinoid is administered to the site of the ocular infection in a subject in need of such treatment or prevention.

According to the invention, there is also provided the manufacture of a topical nasal or inhaled composition comprising a cannabinoid for use in the treatment or prevention of a bacterial infection, wherein between 25 mg and 500 mg of the cannabinoid is administered by a nasal or inhaled dosing regimen to a subject in need of such treatment or prevention.

Preferably use of the composition results in the bacterial decolonisation of the skin and mucosal surface, ocular surface or nasal surface.

Preferably, the use is a dosing regimen applied to the skin and mucosal surfaces (e.g. oral membranes, vaginal membranes, rectal membranes), ocular surfaces or nasal surfaces.

Preferably the bacterium is a biofilm-forming bacterium. Preferably the bacterium is an antibiotic resistant bacterium. The bacterium may be both biofilm-forming and antibiotic resistant.

In a preferred form of the invention, the bacterium is a Gram-positive bacterium, preferably a bacterium species of a genus selected from the list: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof.

Preferably, the cannabinoid is chosen from the list comprising: cannabidiol, cannabinol, cannabigerol, cannabichromene, and Δ⁹-tetrahydrocannabinol. Most preferably, the cannabinoid is cannabidiol.

### Composition

According to another aspect of the invention, there is provided a topical composition comprising a cannabinoid for use in the treatment or prevention of a topical bacterial infection, wherein between 25 mg and 500 mg of the cannabinoid is administered to a subject in need of such treatment or prevention.

According to the invention, there is also provided a topical composition comprising a cannabinoid for administration to the skin and mucus surfaces for use in the treatment or prevention of a topical bacterial infection, wherein between 25 mg and 500 mg of the cannabinoid is administered topically to a subject in need of such treatment or prevention.

According to the invention, there is also provided a topical ocular composition comprising a cannabinoid for use in the treatment or prevention of an ocular bacterial infection, wherein between 25 mg and 500 mg of the cannabinoid is administered to the site of the ocular infection a subject in need of such treatment or prevention.

According to the invention, there is also provided a topical nasal or inhaled composition comprising a cannabinoid for use in the treatment or prevention of a bacterial infection, wherein between 25 mg and 500 mg of the cannabinoid is administered by a nasal or inhaled dosing regimen to a subject in need of such treatment or prevention.

Preferably use of the composition results in the bacterial decolonisation of the skin and mucosal surface, ocular surface or nasal surface.

Preferably, the use is a dosing regimen applied to the skin and mucosal surfaces (e.g. oral membranes, vaginal membranes, rectal membranes), ocular surfaces or nasal surfaces.

Preferably the bacterium is a biofilm-forming bacterium. Preferably the bacterium is an antibiotic resistant bacterium. The bacterium may be both biofilm-forming and antibiotic resistant.

In a preferred form of the invention, the bacterium is a Gram-positive bacterium, preferably a bacterium species of a genus selected from the list: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof.

Preferably, the cannabinoid is chosen from the list comprising: cannabidiol, cannabinol, cannabigerol, cannabichromene, and Δ⁹-tetrahydrocannabinol. Most preferably, the cannabinoid is cannabidiol.

The compositions may contain more than one cannabinoid. For example, the composition of the present invention may contain a combination of two, three or more cannabinoids.

### Cannabinol

The term cannabinoid includes compounds which interact with the cannabinoid receptor and various cannabinoid mimetics, such as certain tetrahydropyran analogs (e.g., Δ⁹-tetrahydrocannabinol, Δ⁸-tetrahydro-cannabinol, 6,6,9-trimethyl-3-pentyl-6H-dibenzo [b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylheptyl,(+)-(3S ,4S)-7-hydroxy-Δ6-tetrahydrocannabinol-1,1-dimethylheptyl, 11-hydroxy-Δ⁹-tetrahydrocannabinol, and Δ8-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate)); certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone); and certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′,6′-hexahydrobiphenyl).

Preferably, the cannabinoid is chosen from the list comprising: cannabidiol, cannabinol, cannabigerol, cannabichromene, and Δ⁹-tetrahydrocannabinol. Most preferably, the cannabinoid is cannabidiol.

Cannabidiol, as used herein, refers to 2-[3-methyl-6-(1-methylethenyl)-2-cyclohexen-1-yl]-5-pentyl-1,3-benzenediol. The synthesis of cannabidiol is described, for example, in Petilka et al., Helv. Chim.Acta, 52: 1102 (1969) and in Mechoulam et al., J. Am. Chem. Soc., 87:3273 (1965), which are hereby incorporated by reference.

The compositions may contain more than one cannabinoid. For example, the composition of the present invention may contain a combination of two, three or more cannabinoids.

Preferably, the composition of the present invention contains a cannabinoid at a concentration of: between 15 mg/mL and 0.1 mg/mL, 10 mg/mL and 1 mg/mL, 8 mg/mL and 2 mg/mL, or 3 mg/mL and 6 mg/mL.

Preferably, the composition of the present invention contains a cannabinoid at a concentration of: 0.1 mg/mL, 0.5 mg/mL, 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL,14.0 mg/mL, 14.5 mg/mL, or 15.0 mg/mL.

Preferably, the composition of the present invention contains a cannabinoid at a concentration of: between 2 mg/mL and 0.1 mg/mL, 1.8 mg/mL and 0.1 mg/mL, 1.5 mg/mL and 0.1 mg/mL, or 1 mg/mL and 0.1 mg/mL.

Preferably, the composition of the present invention contains a cannabinoid at a concentration of: between 2 mg/mL and 1 mg/mL, 1.8 mg/mL and 1 mg/mL, or 1.5 mg/mL and 1 mg/mL.

Preferably, the composition of the present invention contains a cannabinoid at a concentration of: between 0.5% and 35% w/w, 1% and 30% w/w, between 2% and 25% w/w, 0.5% and 20% w/w, between 5% and 20% w/w, between 5% and 15% w/w, between 5% and 10% w/w, between 10% and 20% w/w.

Preferably, the composition of the present invention contains a cannabinoid at a concentration of: 0.5% w/w, 1% w/w, 2.5% w/w, 5% w/w, 10% w/w, 15% w/w or 20% w/w.

### Excipients

The compositions of the present invention may contain excipients to aid in the delivery of the cannabinoid.

The compositions of the present invention may preferably be in the form of ointment vehicles or gel vehicles.

### Ointment vehicles

Ointment vehicles of the present invention preferably contain a mixture of components that can act as both viscosity modifier and solvents for the cannabinoids. Preferably at least one component is a liquid, and at least one component is a solid or semi-solid. The liquid component dissolves the cannabinoid and reduces the viscosity of the ointment. The solid or semi-solid component increases the viscosity of the ointment and may assist in dissolving the cannabinoid. By balancing the amount of each component, a desirable viscosity may be achieved.

Preferred ointment compositions comprise cannabidiol at concentrations of 1-35% w/w, preferably 5-30% w/w, more preferably 10-25% w/w, more preferably 15-20% w/w.

In ointment compositions of the present invention, the active ingredient can be dissolved or dispersed in an ointment base comprising glycols such as polyethylene glycol (PEG). The compositions of the present invention may comprise at least 1% by weight of a poly (substituted or unsubstituted alkylene) glycol or a derivative thereof.

As used herein the term ‘poly (substituted or unsubstituted alkylene) glycol’ refers to polymers having the following repeating unit

—(CH₂)ₙ0-

wherein n is an integer, preferably 2 or 3 and to such polymers wherein one or more methylene groups of each repeating unit is substituted. Suitable substituents include alkoxy groups such as methoxy as in polymethoxypropylene glycol. Such polymers are known by a variety of names, for instance when n=2, as polyethylene glycol, polyoxyethylene, polyoxyethylene glycol and macrogol and, when n=3, as polypropylene glycol, polyoxypropylene and polyoxypropylene glycol. All these are useful in the invention as are derivatives of these polymers.

Suitable derivatives include ethers and esters of the poly (substituted or unsubstituted alkylene) glycols, such as the macrogol ethers and esters, for instance cetomacrogol, glycofurol, the ‘Tweens’ and block copolymers including poly (substituted or unsubstituted alkylene) glycols such as Poloxamers which are block copolymers of polyethylene glycol and polypropylene glycol for instance the ‘Pluronics’, and cross-linked polyethylene glycol. ‘Tween’ and ‘Pluronic’ are trade names for these types of polymer.

The poly (substituted or unsubstituted alkylene) glycols and derivatives thereof may be used singly or various grades and types may be used in combination to achieve the desired physical properties of the composition.

Preferably the composition comprises polyethylene glycol (PEG) or a derivative thereof.

The PEG base can comprise PEG of a single molecular weight grade, or a mixture of one or more molecular weight grades. Representative PEG molecular weight grades include PEG 200, PEG 300, PEG 400, PEG 540, PEG 600, PEG 800, PEG 900, PEG 1000, PEG 1450, PEG 1600, PEG 3000, PEG 3350, PEG 4000, PEG 4500, PEG 6000, PEG 8000, and PEG 20000. The PEG used in embodiments of the invention preferably comprises at least a lower molecular weight polyethylene glycol such that the composition has good spreading properties at ambient and body temperatures. Generally, PEG200-PEG600 are liquids at 200C and PEGs with a MW>600 are semi-solid to solid at 200C.

Other embodiments of the invention comprise active ingredient dissolved or dispersed in an ointment base of propylene glycol, dipropylene glycol, polypropylene glycol (PPG), or mixtures thereof. Representative PPG molecular weight grades include PPG 200, PPG 400, PPG 425, PPG 750, PPG 1200, PPG 2000, PPG 3000, and PPG 4000. Preferred compositions comprise PPG of molecular weight 2000 or higher. Other embodiments comprise active ingredient dissolved or dispersed in butylene glycol or hexylene glycol.

Other embodiments of the invention comprise mixtures of any of the aforementioned polyethylene glycols, polypropylene glycols, propylene glycol, dipropylene glycol, butylene glycol, and hexylene glycol. For example, it may be preferable to use a mixture of PEGs with a MW between 200-600 and PEGs with a MW above 1000. By varying the ratio of liquid and solid PEG components, a composition with a desirable viscosity can be generated for different situations and application sites.

Examples of suitable components include Polyethylene glycol 400 as the liquid component of the vehicle and Polyethylene glycol 4000 as the solid or semi-solid component of the vehicle.

Polyethylene Glycols

| Liquids | Semisolids | Hard solids |
|---|---|---|
| PEG 200 | PEG 1000 | PEG 4000 |
| PEG 300 | PEG 1540 | PEG 6000 |
| PEG 400 | | |

Polyethylene Glycol Derivatives

| Derivative | Chemical composition | Consistency |
|---|---|---|
| Glycofurol | Tetrahydrofurfuryl alcohol | Liquid |
| | polyethylene glycol ether | |
| Tween 60 | Poloxyethylene sorbitan | Semi-solid |
| | monostearate | |
| Tween 80 | Poloxyethylene sorbitan | Liquid |
| | monooleate | |

Preferably, the liquid and semi-solid or solid components of the ointment vehicles will have a lipophilicity similar to that of PEG 400 and/or PEG 4000. It has been noted that the inclusion of petrolatum did not provide an effective ointment for the delivery of cannabinoids. This may be due to the highly lipophilic nature of petrolatum, which caused the cannabinoid to preferentially partition in the composition and not move into the skin or mucosal surface, ocular surface or nasal surface. It is desirable to include components in the ointment vehicle that allow the cannabinoid to preferentially partition into the skin.

### Gel Vehicles

Gel vehicles of the present invention preferably contain a volatile solvent to dissolve the cannabinoid, and a viscosity modifier to increase the viscosity.

By using a volatile solvent, one can achieve much higher, non-crystalline (i.e., in solution), concentrations of cannabinoids. The cannabinoids can be dissolved in much higher concentrations of the volatile solvent, and then once applied to the skin and the volatile solvent has evaporated, the cannabinoids remain on the skin in high concentrations. The volatile solvent may, for example, be a C₂₋₆ low molecular weight alcohol such as methanol, isopropanol, propanol, 2-butanol, n-butanol or ethanol. Alternatively, the volatile solvent may be a siloxane. Other suitable volatile solvents will be clear to the skilled reader.

In a preferred form of the invention, the composition comprises a combination of a C₂₋₆ low molecular weight alcohol and a siloxane.

Advantageously, in some embodiments, the volatile solvent is a liquid at ambient temperatures. Preferably the volatile solvent is liquid at about 30° C., or less, or at about 25° C. Preferably the level of volatility of the volatile solvent is about the same as that of isopropyl alcohol. Preferably, the boiling point of the volatile solvent is between about 70° C. and 110° C. at atmospheric pressure. Preferably, the boiling point of the volatile solvent is between about 80° C. and 105° C. at atmospheric pressure. Preferably, the boiling point of the volatile solvent is between about 85° C. and 105° C. at atmospheric pressure.

Preferred gel compositions comprise cannabidiol at concentrations of 1-35% w/w, preferably 5-30% w/w, more preferably 10-25% w/w, more preferably 15-20% w/w.

In gel compositions of the present invention, the active ingredient is dissolved or dispersed in a gel base comprising a volatile silicone liquid, preferably a non-polymeric siloxane. Preferred silicone liquids have viscosities in the range from about 0.5 cSt to about 5 cSt. A preferred silicone is hexamethyldisiloxane (HDS) having a viscosity of approximately 0.65 cSt. Other preferred siloxanes include trimethylsiloxane, cyclotetrasiloxane, cyclopentasiloxane, cyclohexasiloxane, and lower molecular weight dimethicones of viscosities ≤10 cSt. Compositions of the present invention may also comprise mixtures of volatile silicones. Preferred volatile silicones have heats of vaporization at 25° C.<500 kJ/kg, more preferably <400 kJ/kg, more preferably <300 kJ/kg, and more preferably <200 kJ/kg.

In a preferred form of the invention, the siloxane contains from one to eight silicon atoms per molecule. In a preferred form of the invention, the siloxane contains from two to five silicon atoms per molecule. In one embodiment, the siloxane contains two or three silicon atoms.

The siloxanes may have between one and eight methyl groups. In one embodiment, the siloxane is selected from the group consisting of: hexamethyldisiloxane, octamethyltrisiloxane and combinations thereof. These are the most volatile siloxanes and are thus the most advantageous. Preferably the level of volatility of the siloxane is about the same as that of isopropyl alcohol.

In another embodiment, the siloxane contains 4 or 5 silicon atoms, and is, for example, decamethyltetrasiloxane or dodecamethylpentasiloxane. In another embodiment, the siloxane is a cyclical 4 or 5 silicon atom compound such octamethylcyclotetrasiloxane (CAS# 556-67-2) or decamethylcyclopentasiloxane (CAS# 541-02-6).

In one form of the invention, the volatile solvent is hexylmethyldisiloxane which is combined with less volatile polymethylsiloxane.

Advantageously, in some embodiments, the volatile solvent is selected from the group consisting of: C₂₋₆ alcohols, and combinations thereof. Advantageously, in some embodiments, the volatile solvent is selected from the group consisting of: C₂₋₄ alcohols, and combinations thereof. In specific embodiments, the volatile solvent is selected from the group consisting of: ethyl alcohol (or ethanol), n-propanol, isopropyl alcohol, butanol, and combinations thereof. Other volatile solvents will be clear to the skilled reader.

In a preferred form of the invention, the composition comprises a combination of a C₂₋₆ low molecular weight alcohol and a non-polymeric siloxane.

Gel compositions preferably also comprise a cosolvent which does not have significant volatility at room or body temperatures. Preferred cosolvents comprise glycols and their derivatives. Representative cosolvents comprise diethylene glycol monoethyl ether (Transcutol®), polypropylene glycol stearyl ether (Arlamol™ PS11E), propylene glycol diacetate, propylene glycol dicaprylate, propylene glycol monolaurate, propylene glycol monopalmitostearate, propylene glycol monostearate, and mixtures thereof.

Preferably the cosolvent has a boiling point @760.00 mm Hg between 160° C. and 500° C. For example, the cosolvent preferably has a minimum boiling point @760.00 mm Hg of at least 160° C., at least 165° C., at least 170° C., at least 175° C., at least 180° C., at least 185 ° C., or at least 190° C. The cosolvent preferably has a maximum boiling point @760.00 mm Hg of at most 500° C., at most 495° C., at most 490° C., at most 485° C., at most 480° C., at most 475° C., at most 470° C., or at most 465° C.

Gel compositions may also comprise non-volatile ingredients that increase the composition viscosity and/or result in improved skin feel; i.e. emollients. The viscosity modifier in the gel compositions of the present invention serves to increase the viscosity of the gel. As volatile solvents are generally liquids, a thickening agent is required to keep the gel on the skin, mucosal surface etc for a desirable length of time. Representative ingredients include higher molecular weight dimethicones with viscosities ranging from about 100 cSt to about 12,500 cSt, polyethylene glycol/polypropylene glycol dimethicones such as PEG/PPG-19/19 dimethicone (DOWSIL™ BY 11-030), PEG/PPG-18/18 dimethicone, dimethiconol, dimethiconol/trimethylsiloxysilicate crosspolymers, and derivatives thereof.

The compositions of the present invention may contain water (aqueous) or may be non-aqueous.

The formulations of the present invention may be presented as, for instance, ointments, creams or lotions, eye ointments and eye or ear drops, impregnated dressings, and may contain appropriate conventional additives such as preservatives, solvents to assist drug penetration and emollients in ointments and creams. The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

The composition of this invention may also include minor amounts of conventional additives such as viscosity modifiers, for example xanthan gum, and preservatives, such as phenoxyethanol or benzyl alcohol, including mixtures thereof. For some therapeutic agents it may be necessary to incorporate buffering agents to maintain a suitable pH.

Suitable preservatives for use in such a composition or medicament include, for example, phenoxyethanol, and other preservatives conventionally used in pharmaceutical preparations, especially in creams. Suitable preservatives include methyl hydroxybenzoate, chlorocresol, sorbic acid and benzoic acid.

The compositions of the invention may be produced by conventional pharmaceutical techniques. Thus, ointments and creams are conveniently prepared by mixing together at an elevated temperature, preferably 60-70° C., the components constituting the vehicle until an emulsion has formed. The mixture may then be cooled to room temperature, and, after addition of the cannabinoid, together with any other ingredients, stirred to ensure adequate dispersion.

Liquid preparations, such as ear and eye drops, are produced by dissolving the therapeutic agent in the components constituting the vehicle and the other ingredients are then added. The resulting solution or suspension is distributed into glass or plastic bottles or in single dose packs such as soft gelatine capsules which are then heat sealed.

Compositions of the invention are intended for pharmaceutical or veterinary use.

The invention encompasses variations on the above composition, as the amounts of the respective compounds may vary by±5%, ±7.5%, ±10%, ±15%, ±17.5%, or ±20%.

The present invention encompasses compositions wherein the relative proportions of the active ingredient and/or each excipient independently vary from those specified above. In one form of the invention, the relative proportions of the active ingredient and/or each excipient independently vary by up to 50% from those specified above. In one form of the invention, the relative proportions of the active ingredient and/or each excipient independently vary by up to 40% from those specified above. In one form of the invention, the relative proportions of the active ingredient and/or each excipient independently vary by up to 30% from those specified above. In one form of the invention, the relative proportions of the active ingredient and/or each excipient independently vary by up to 20% from those specified above. In one form of the invention, the relative proportions independently vary by up to 10% from those specified above. In one form of the invention, the relative proportions of the active ingredient and/or each excipient independently vary by up to 5% from those specified above. In one form of the invention, the relative proportions independently vary by up to 10% from those specified above. In one form of the invention, the relative proportions of the active ingredient and/or each excipient independently vary by up to 2% from those specified above.

As would be understood by a person skilled in the art, the sum of the percentages of the excipients and the active cannot exceed 100, and the variations described above are subject to this limitation. As would be understood by a person skilled in the art, the sum of the percentages of the excipients and the active may be less than 100, as forms of the invention include components other than those specified.

The variation described above is a percentage variation of a relative proportion. By way of example, a 20% variation of the relative proportion of a component (excipient or active) that is specified at 1% means that the relative proportion of that component may be 0.8-1.2%.

### Treatment

The term “infection” as used herein means colonization by a micro-organism and/or multiplication of a micro-organism, in particular, a bacterium and more particularly a biofilm-forming bacterium. The infection may be unapparent or result in local cellular injury. The infection may be localized, subclinical and temporary or alternatively may spread by extension to become an acute or chronic clinical infection. The infection may also be a latent infection, in which the microorganism is present in a subject, however the subject does not exhibit symptoms of disease associated with the organism.

Preferably the composition of the present invention delivers between 25 mg and 500 mg of the cannabinoid to the subject.

The phrase “therapeutically effective amount” as used herein refers to an amount of the cannabinoid sufficient to inhibit bacterial growth associated with bacterial carriage or a bacterial infection. That is, reference to the administration of the therapeutically effective amount of a cannabinoid according to the methods or compositions of the invention refers to a therapeutic effect in which substantial bacteriocidal or bacteriostatic activity causes a substantial inhibition of the relevant bacterial carriage or bacterial infection. The term “therapeutically effective amount” as used herein, refers to a nontoxic but sufficient amount of the composition to provide the desired biological, therapeutic, and/or prophylactic result. The desired results include elimination of bacterial colonization or reduction and/or alleviation of the signs, symptoms, or causes of a disease, or any other desired alteration of a biological system. An effective amount in any individual case may be determined by one of ordinary skill in the art using routine experimentation. In relation to a pharmaceutical composition, effective amounts can be dosages that are recommended in the modulation of a diseased state or signs or symptoms thereof. Effective amounts differ depending on the pharmaceutical composition used and the route of administration employed. Effective amounts are routinely optimized taking into consideration various factors of a particular subject, such as age, weight, gender, etc. and the area affected by disease or disease-causing microorganisms.

As used herein, “treating” or “treatment” refers to inhibiting the disease or condition, i.e., arresting or reducing its development or at least one clinical or subclinical symptom thereof, for example reducing or eliminating a bacterial infection. “Treating” or “treatment” further refers to relieving the disease or condition, i.e., causing regression of the disease or condition or at least one of its clinical or subclinical symptoms. The benefit to a subject to be treated is either statistically significant or at least perceptible to the subject and/or the physician. In the context of treating a bacterial infection, the term treatment includes reducing or eliminating colonization by bacteria and/or multiplication of bacteria, including reducing biofilm formation or disrupting existing biofilms.

Decolonization, or bacterial decolonization, is the reduction or elimination of the presence of a bacteria such as an antimicrobial resistant pathogen (for example methicillin-resistant Staphylococcus aureus (MRSA)) in a subject. By pre-emptively treating subjects who are colonized with, for example, an antimicrobial resistant organism prior to an event such as surgery, the likelihood of the subject going on to develop a life-threatening health care-associated infection is reduced. Common sites of bacterial colonization include the nasal passage, skin including the skin of the groin, and the oral cavity.

In one form of the invention, reducing or eliminating colonization by bacteria means reducing or eliminating colonization by bacteria as measured by % bacteria killed. For example, the % bacteria killed may be 50%, 60%, 70%, 80%, 90%, 95% or 97%.

In one form of the invention, reducing or eliminating colonization by bacteria means reducing or eliminating colonization by bacteria as measured by a log₁₀ reduction in bacterial numbers. For example, the log₁₀ reduction in bacteria may be by one log₁₀ reduction, by two log₁₀ reduction, by three log₁₀ reduction, by four log₁₀ reduction, by five log₁₀ reduction, by six log₁₀ reduction, by seven log₁₀ reduction or more.

The term a “preventative effective amount” as used herein means an amount of the composition, which when administered according to a desired dosage regimen, is sufficient to at least partially prevent or delay the onset of the microbial infection.

### Topical Infections

In one aspect, the composition used in the treatment regimen is a topical pharmaceutical composition for the treatment of an infection of a dermal or mucosal surface.

In one form of the invention, the infection is related to one or more of the following conditions: acne, rash, blisters, burns, itch, cellulitis, folliculitis, nail infections, boils, hair infections, scalp infections, impetigo, haemorrhoids, canker sore, gingivitis, periodontitis, vaginitis, nose lesions, swelling, cut, surgical incision, sunburn, cracked skin, and combinations thereof.

In one form of the invention, the infection is an acute bacterial skin and skin structure infection (ABSSSI) where the infection is related to one or more of the following conditions: cellulitis/erysipelas, wound infection, and major cutaneous abscess that have a minimum lesion surface area of approximately 75 cm².

In one form of the invention, the infection is a complicated skin and skin structure infection (cSSSI) where the infection involves deep subcutaneous tissues or needs surgery in addition to antimicrobial therapy.

In one form of the invention, the infection is a non-complicated or community acquired skin or skin structure infection.

The topical treatment regimen may comprise the administration of between 25 mg and 500 mg of a cannabinoid directly to a dermal or mucosal surface of the subject. Preferably, the cannabinoid is applied topically to the skin or mucosal membranes (oral, vaginal, rectal) of the subject. The use may comprise administering between 25 mg and 500 mg of a cannabinoid to the skin or mucosal membranes (oral, vaginal, rectal) of a subject.

### Ocular Infections

In one aspect, the composition used in the treatment regimen is an ocular pharmaceutical composition for the treatment of an infection of an ocular infection.

Ocular infections can be divided into (i) infections affecting the cornea and conjunctiva; (ii) infections in the soft tissue surrounding the eye (ocular adnexa and orbit) which can involve the eye indirectly and can spread from the orbit into the brain; and (iii) infections inside the eye (endophthalmitis), often following penetrating ocular trauma or after intraocular surgery. All the above infections may be treated by the present regimen of cannabinoid delivery.

The ocular treatment regimen may comprise the administration of between 25 mg and 500 mg of a cannabinoid directly to an ocular surface of the subject. Preferably, the cannabinoid is applied topically to the eye of the subject. However, the cannabinoid dosing regimen may comprise administering the cannabinoid via intraocular injection, scleral injection, slow release implant or other delivery method. The use may comprise administering between 25 mg and 500 mg of a cannabinoid to the eye of a subject.

### Infections Treated by Nasal or Pulmonary Administration

In one aspect, the composition used in the treatment regimen is a nasal or pulmonary pharmaceutical composition for the treatment of an infection. Any infection in a subject by a bacteria may be treated using a nasal or pulmonary delivered treatment regime.

Preferably, infections of the nasal cavity, sinuses, respiratory tract and lungs are treated using a nasal or pulmonary treatment regime. For example, the treatment regimen of the present invention may be used to treat: pneumonia; sinus infection; infections associated with cystic fibrosis; infections associated with asthma; infections associated with acute respiratory distress syndrome (ARDS); infections associated with pneumoconiosis; infections associated with interstitial lung disease (ILD).The nasal or pulmonary treatment regimen may comprise the administration of between 25 mg and 500 mg of a cannabinoid to the nasal or pulmonary system of the subject. The cannabinoid may enter the blood stream via absorption in the nasal or pulmonary system and be systemically available to the subject. However, the cannabinoid dosing regimen may alternatively comprise administering the cannabinoid to the nasal or pulmonary system for a localised topical effect. The use may comprise nasal or pulmonary administration of between 25 mg and 500 mg of a cannabinoid to a subject.

### Biofilm Disruption

It is believed that the treatment regimens of the present invention can disrupt or prevent the formation of biofilms. Bacterial infections may result in the formation of biofilms in the subject, for example in the lungs, on the skin or in the GI tract. Such biofilm-associated infections are often difficult to treat.

Without being held to any theory, we believe the cannabinoids are capable of interfering with the biofilm forming activity of a biofilm-forming bacterium, thereby rendering it more susceptible to the antibacterial activity of the cannabinoid.

The term “biofilm-forming bacterium” as used herein means a bacterium that forms a biofilm, where a biofilm is an aggregate of microorganisms in which cells are embedded in a self-produced matrix of extracellular polymeric substances that are adherent to each other, and/or a surface; and/or a microbially-derived, sessile community characterised by cells attached to a substratum, interface or to each other, and are embedded in a matrix of extracellular polymeric substances (EPS) that they have produced.

The compositions of the present invention biofilm may disrupt an already existing biofilm, or may reduce or prevent the formation of a biofilm.

When an existing biofilm is disrupted, the bacteria in the biofilm may be subject to one or more of the following effects:
killing of the bacteria within the biofilm;
reduction in growth of the bacteria within the biofilm;
a reduction in the adherence of the bacteria to the surface on which the biofilm has formed;
a reduction in the rate of formation of the extracellular polymeric substance (EPS) matrix;
a reduction in the viscosity of the EPS matrix.

When inhibition of biofilm formation occurs, the bacteria in the biofilm may be subject to one or more of the following effects:
killing of the bacteria that would form the biofilm prior to or during biofilm formation;
reduction in growth of the bacteria that would form the biofilm prior to or during biofilm formation;
a reduction in the adherence of the bacteria to the surface on which the biofilm will be formed;
a reduction in the rate of formation of the extracellular polymeric substance (EPS) matrix during biofilm formation;
a reduction in the viscosity of the EPS matrix during biofilm formation.

Preferably, the treatment regimens of the present invention cause an inhibition of biofilm growth wherein the OD590 demonstrates a ≥70% growth inhibition compared to a growth control. An example of this measurement is provided in the Examples of the present specification.

### Bacterium

Preferably, the bacterium of any of the aspects of the present invention is a Gram-positive bacterium.

In a preferred form of the invention, the bacterium is a bacterium species of a genus selected from the list: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof.

In a preferred form of the invention, the bacterium is a bacterium species of a genus selected from the following genus: Staphylococcus spp., Streptococcus spp., Bacillus spp., Kocuria spp., and Enterococcus spp..

In a preferred form of the invention, the bacterium is selected from the following species: Staphylococcus aureus (including MRSA), Staphylococcus warneri, Staphylococcus lugdunensis, Staphylococcus epidermidis, Staphylococcus pyogenes, Staphylococcus capitis, Streptococcus pneumoniae, Streptococcus pyogenes, Bacillus cereus, Bacillus megaterium, Bacillus subtilis, Enterococcus faecium, Enterococcus faecalis, Corynebacterium jeikeium, Kocuria rosea, and Propionibacterium acnes.

In a preferred form of the invention, the bacterium is selected from the following species: Staphylococcus aureus (including MRSA), Staphylococcus warneri, Staphylococcus capitis, Staphylococcus epidermidis, Streptococcus pneumoniae, Streptococcus pyogenes, Bacillus cereus, Bacillus megaterium, Bacillus subtilis, Enterococcus faecium, Kocuria rosea, and Enterococcus faecalis.

More preferably the bacterium is a bacterium other than Staphylococcus aureus or methicillin-resistant Staphylococcus aureus.

In one form of the invention, the bacterium is MSRA.

In one form of the invention, the infection is a disease to be treated in a non-human subject and may be selected from swine dysentery; leptospirosis in cattle, pigs, horses and dogs; infections of the skin; pyodermas in dogs; otitis externa; mastitis in cattle, sheep and goats; streptococcal mastitis; streptococcal infection in horses, in pigs and in other animal species; pneumococcal infection in calves and in other animal species; glanders; conjunctivitis; enteritides; pneumonias; brucellosis in cattle, sheep and pigs; atrophic rhinitis in pigs; septicaemias; metritis-mastitis-agalactia (MA) Syndrome; Klebsiella infections; pseudotuberculosis; infectious pleuropneumonia; primary pasteurelloses; joint ill; necrobacillosis in cattle and in domestic animals; leptospirosis; erysipelas in pigs and other animal species, listeriosis; anthrax, clostridioses; tetanus infections, botulism; infections with Corynebacterium pyogenes; tuberculosis in cattle, sheep and other animal species; paratuberculosis in ruminants; nocardiosis; Q fever; ornithosis-psittacosis; encephalomyelitis; mycoplasmosis in cattle and other animals; enzootic pneumonia in pigs.

The topical administration may comprise the administration of the therapeutically effective amount of a cannabinoid directly to a dermal or mucosal surface of the subject. Preferably, the cannabinoid is applied topically to the skin, mucosal membranes (oral, nasal, vaginal, rectal) or eye of the subject. The use may comprise administering a therapeutically effective amount of a cannabinoid to the skin, mucosal membranes (oral, nasal, vaginal, rectal) or eye of a subject.

### Additional Antimicrobials

Other active agents may also be incorporated into the composition of the present invention. For example, additional antimicrobial agents such as antibacterials, antifungals etc may be incorporated.

For example, the composition may further comprise benzoyl peroxide, erythromycin, clindamycin, doxycycline or meclocycline.

Additional antimicrobial agents that can be used include, but are not limited to silver compounds (e.g., silver chloride, silver nitrate, silver oxide), silver ions, silver particles, iodine, povidone/iodine, chlorhexidine, 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin (s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, vancomycin, azaserine, candicidin(s), chlorphenesin, dermostatin(s), filipin, fungichromin, mepartricin, nystatin, oligomycin(s), ciproflaxacin, norfloxacin, ofloxacin, pefloxacin, enoxacin, rosoxacin, amifloxacin, fleroxacin, temafloaxcin, lomefloxacin, perimycin A or tubercidin, and the like.

### Subject

The subject may be any subject capable of infection by a bacteria. The subject may be mammalian or avian. Preferably, the subject is selected from the group comprising human, canine, avian, porcine, bovine, ovine, equine, and feline. Most preferably, the subject is selected from the group comprising human, bovine, porcine, equine, feline and canine. Most preferably, the subject is human.

### Dosing

Preferably the total daily dose administered by the topical dosing regimen of the present invention is between 25 mg and 500 mg cannabinoid.

Preferably, the total daily dose administered by the dosing regimen of the present invention is:
between 25 mg and 500 mg cannabinoid when delivered topically;
between 25 mg and 500 mg cannabinoid when delivered via ocular delivery;
between 25 mg and 500 mg cannabinoid when delivered via nasal or pulmonary delivery.

In certain embodiments, the total daily dose of cannabinoid administered by the dosing regimen of the present invention has a lower limit selected from the group consisting of: 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, 150 mg, 160 mg, 170 mg, 200 mg, 210 mg, 220 mg, 230 mg, 240 mg, 250 mg, 260 mg, 270 mg, 280 mg, 290 mg, 300 mg, 320 mg, 350 mg, 400 mg, 500 mg, 600 mg, 700 mg, 800 mg, 900 mg, 1000 mg, 1500 mg and 1900 mg; and an upper limit selected from the group consisting of: 30 mg, 50 mg, 70 mg, 100 mg, 150 mg, 200 mg, 210 mg, 220 mg, 230 mg, 240 mg, 250 mg, 260 mg, 270 mg, 280 mg, 290 mg, 300 mg, 320 mg, 350 mg, 400 mg, 500 mg, 600 mg, 700 mg, 800 mg, 900 mg, 1000 mg, 1500 mg and 2000 mg. Preferably total daily dose of cannabinoid administered by the dosing regimen of the present invention is between 25 mg and 500 mg, between 50 mg and 400 mg, between 100 mg and 250 mg.

In one embodiment of the invention, the cannabinoid is administered to the subject using a dosing regimen selected from the group consisting of: three times daily; two times daily; daily; every second day, every third day, once weekly; once fortnightly and once monthly.

For example, if the cannabinoid is administered for the purpose of topical nasal decolonising, about 200 mg per day is administered in two doses of about 100 mg each (50 mg to each nare in each administration).

In accordance with certain embodiments, the composition is administered regularly until treatment is obtained. In one preferred embodiment, the composition is administered to the subject in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention. Preferably, the composition of the treatment regimen is administered to the subject between 1 and 5 times per day, more preferably once or twice per day.

The compositions used in the topical treatment regimens of the invention may be prepared for oral, inhaled (pulmonary), nasal, ocular, or any other form of administration. Preferably the compositions are administered, for example, ophthalmically, buccal, rectally, vaginally, intranasally or by aerosol administration.

The mode of administration is preferably suitable for the form in which the composition has been prepared. The mode of administration for the most effective response may be determined empirically and the means of administration described below are given as examples, and do not limit the method of delivery of the composition of the present invention in any way. All the above compositions are commonly used in the pharmaceutical industry and are commonly known to suitably qualified practitioners.

The compositions of the invention may optionally include pharmaceutically acceptable nontoxic excipients and carriers. As used herein, a “pharmaceutical carrier” is a pharmaceutically acceptable solvent, suspending agent, excipient or vehicle for delivering the compounds to the subject. The carrier may be liquid or solid and is selected with the planned manner of administration in mind.

The composition of the invention may be selected from the group consisting of: an immediate release composition, a delayed release composition, a controlled release composition and a rapid release composition.

The composition of the invention may further comprise an anti-inflammatory agent (such as a corticosteroid). If the composition is a topical composition, an anticomedolyic agent (such as tretinoin), and/or a retinoid or derivative thereof may also be added.

The compositions described herein may be formulated by including such dosage forms in an oil-in-water emulsion, or a water-in-oil emulsion. In such a composition, the immediate release dosage form is in the continuous phase, and the delayed release dosage form is in a discontinuous phase. The composition may also be produced in a manner for delivery of three dosage forms as hereinabove described. For example, there may be provided an oil-in-water-in-oil emulsion, with oil being a continuous phase that contains the immediate release component, water dispersed in the oil containing a first delayed release dosage form, and oil dispersed in the water containing a third delayed release dosage form.

The compositions described herein may be in the form of a liquid composition. The liquid composition may comprise a solution that includes a therapeutic agent dissolved in a solvent. Generally, any solvent that has the desired effect may be used in which the therapeutic agent dissolves and which can be administered to a subject. Generally, any concentration of therapeutic agent that has the desired effect can be used. The composition in some variations is a solution which is unsaturated, a saturated or a supersaturated solution. The solvent may be a pure solvent or may be a mixture of liquid solvent components. In some variations the solution formed is an in-situ gelling composition. Solvents and types of solutions that may be used are well known to those versed in such drug delivery technologies.

The composition may or may not contain water. Preferably, the composition does not contain water, i.e. it is non-aqueous. In another preferred embodiment, the composition does not comprise a preservative.

The administration of the cannabinoids in accordance with the methods and compositions of the invention may be by any suitable means that results in an amount sufficient to treat a microbial infection or to reduce microbial growth at the location of infection.

The cannabinoid may be contained in any appropriate amount and in any suitable carrier substance and is generally present in an amount of 1-95% by weight of the total weight of the composition.

The pharmaceutical or veterinary composition may be formulated according to the conventional pharmaceutical or veterinary practice (see, for example, Remington: The Science and Practice of Pharmacy, 20th edition, 2000, ed; A. R. Gennaro, Lippincott Williams & Wilkins, Philadelphia, and Encyclopedia of Pharmaceutical Technology, eds; J. Swarbrick and J. C. Boylan, 1988-1999, Marcel Dekker, New York; Remington's Pharmaceutical Sciences, 18^{th} Edition, Mack Publishing Company, Easton, Pa., USA).

Generally, examples of suitable carriers, excipients and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

The composition may be in the form of a controlled-release composition and may include a degradable or non-degradable polymer, hydrogel, organogel, or other physical construct that modifies the release of the cannabinoid. It is understood that such compositions may include additional inactive ingredients that are added to provide desirable colour, stability, buffering capacity, dispersion, or other known desirable features. Such compositions may further include liposomes, such as emulsions, foams, micelles, insoluble monolayers, liquid crystals, phospholipid dispersions, lamellar layers and the like. Liposomes for use in the invention may be formed from standard vesicle-forming lipids, generally including neutral and negatively charged phospholipids and a sterol, such as cholesterol.

### Topical Compositions

Compositions of the invention may be administered topically. Therefore, contemplated for use herein are compositions adapted for the direct application to the skin. Preferably, the topical composition comprises between 25 mg and 500 mg of a cannabinoid.

The composition may be in a form selected from the group comprising suspensions, emulsions, liquids, creams, oils, lotions, ointments, gels, hydrogels, pastes, plasters, roll-on liquids, skin patches, sprays, glass bead dressings, synthetic polymer dressings and solids. For instance, the compositions of the invention may be provided in the form of a water-based composition or ointment which is based on organic solvents such as oils. Alternatively, the compositions of the invention may be applied by way of a liquid spray comprising film forming components and at least a solvent in which the cannabinoids are dispersed or solubilised.

The composition of the invention may be provided in a form selected from the group comprising, but not limited to, a rinse, a shampoo, a lotion, a gel, a leave-on preparation, a wash-off preparation, and an ointment.

Various topical delivery systems may be appropriate for administering the compositions of the present invention depending up on the preferred treatment regimen. Topical compositions may be produced by dissolving or combining the cannabinoids of the present invention in an aqueous or non-aqueous carrier. In general, any liquid, cream, or gel or similar substance that does not appreciably react with the compound or any other of the active ingredients that may be introduced into the composition and which is non-irritating is suitable. Appropriate non-sprayable viscous, semi-solid or solid forms can also be employed that include a carrier compatible with topical application and have dynamic viscosity preferably greater than water.

Suitable compositions are well known to those skilled in the art and include, but are not limited to, solutions, suspensions, emulsions, creams, gels, ointments, powders, liniments, salves, aerosols, transdermal patches, etc., which are, if desired, sterilised or mixed with auxiliary agents, e.g. preservatives, stabilisers, emulsifiers, wetting agents, fragrances, colouring agents, odour controllers, thickeners such as natural gums, etc. Particularly preferred topical compositions include ointments, creams or gels.

Ointments generally are prepared using either (1) an oleaginous base, i.e., one consisting of fixed oils or hydrocarbons, such as white petroleum, mineral oil, or (2) an absorbent base, i.e., one consisting of an anhydrous substance or substances which can absorb water, for example anhydrous lanolin. Customarily, following formation of the base, whether oleaginous or absorbent, the cannabinoids are added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface-active agent, such as sodium lauryl sulfite; hydrophilic colloids, such as acacia colloidal clays, veegum and the like. Upon formation of the emulsion, the cannabinoids can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the cannabinoids are added to the composition at the desired concentration at a point preceding addition of the gelling agent.

The amount of antibiotic compounds incorporated into a topical composition is not critical; the concentration should be within a range sufficient to permit ready application of the composition such that an effective amount of the cannabinoids is delivered.

### Ocular Compositions

Compositions of the invention may be administered via topical ocular delivery. Preferably, the ocular composition comprises between 100 mg and 500 mg of a cannabinoid.

Ocular delivery encompasses delivery to the sclera, retina, intraocular fluid, tissue surrounding the eyeball. For example, the delivery may be topical delivery (creams, gels, ointments, sprays, eye drops), intraocular implant or other means.

Artificial tear vehicles may be used for ocular cannabinoid delivery. More viscous artificial tears use high concentrations of viscosity enhancing agents, such as Celluvisc®, high viscosity carboxymethyl cellulose (CMC) and Refresh Liquigel®, a blend of 0.35% high viscosity CMC and 0.65% low viscosity CMC.

Gelling agents may be used for cannabinoid delivery. Such agents may be instilled as liquid and then almost immediately triggered to a gel phase. Timoptic gel (gellan gum), AzaSite® (polycarbophil, poloxamer), and Besivance®, (polycarbophil, poloxamer), 0.3% alginate Keltrol® are examples of such agents. Another gelling agent is polycarbophil-poloxamer gels (eg Durasite®).

Ocular delivery may also comprise injecting the cannabinoid into the sclera, intraocular space or into the area behind the eye. Compositions suitable for ocular injection optionally include sterile aqueous solutions (where water-soluble) or dispersions and sterile powders for the extemporaneous preparation of sterile injectable solutions or dispersion. Alternatively, the compounds of the invention are, in certain aspects encapsulated in liposomes and delivered in injectable solutions to assist their transport across cell membrane. Alternatively, or in addition, such preparations contain constituents of self-assembling pore structures to facilitate transport across the cellular membrane. The carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by the use of a coating such as lecithin, by the maintenance of the required particle size in the case of dispersion and by the use of surfactants. Prolonged absorption of the injectable compositions is in certain aspects brought about by the use in the compositions of agents delaying absorption, for example, aluminum monostearate and gelatin.

### Nasal and Pulmonary Compositions

Compositions of the invention may be administered via topical nasal or pulmonary delivery. Preferably, the nasal or pulmonary composition comprise between 25 mg and 500 mg of a cannabinoid.

A wide range of mechanical devices designed for pulmonary delivery of therapeutic products exist, including but not limited to nebulizers, metered-dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of compositions suitable for the dispensing of the cannabinoid. Typically, each composition is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Compositions suitable for use with a nebulizer, either jet or ultrasonic, will typically comprise the cannabinoid suspended in water or non-aqueous solvent. The composition may also include a buffer and a simple sugar (e.g., for stabilization and regulation of osmotic pressure). The nebulizer composition may also contain a surfactant, to reduce or prevent surface induced aggregation of the cannabinoid caused by atomization of the solution in forming the aerosol.

Compositions for use with a metered dose inhaler device will generally comprise a finely divided powder containing the cannabinoid suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Compositions for dispensing from a powder inhaler device will comprise a finely divided dry powder containing the cannabinoid and may also include a bulking agent, such as lactose, sorbitol, sucrose, or mannitol in amounts which facilitate dispersal of the powder from the device, e.g., 50 to 90% by weight of the composition. The cannabinoid should most advantageously be prepared in particulate form with an average particle size of less than 10 microns, most preferably 0.5 to 5 microns, for most effective delivery to the distal lung.

Nasal delivery of cannabinoids in the treatment regimes of the present invention is also contemplated. Nasal delivery allows the passage of the cannabinoid to the blood stream directly after administering the therapeutic product to the nose, without the necessity for deposition of the cannabinoid in the lung. Compositions for nasal delivery include those with dextran or cyclodextran.

### General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference, which means that it should be read and considered by the reader as part of this text. That the document, reference, patent application or patent cited in this text is not repeated in this text is merely for reasons of conciseness. No admission is made that any of the references constitute prior art or are part of the common general knowledge of those working in the field to which this invention relates.

Throughout this specification, unless the context requires otherwise, the term antimicrobial is understood to include compounds with antibacterial properties.

Throughout this specification, unless the context requires otherwise, the word “comprise”, or variations such as “comprises” or “comprising”, will be understood to imply the inclusion of a stated integer or group of integers but not the exclusion of any other integer or group of integers.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino) ethane salt, monomethyl-monoethanolamine salt, procaine salt and caffeine salt), a basic amino acid salt (such as arginine salt and lysine salt), tetraalkyl ammonium salt and the like, or other salt forms that enable the pulmonary hypertension reducing agent to remain soluble in a liquid medium, or to be prepared and/or effectively administered in a liquid medium, preferable an aqueous medium. The above salts may be prepared by a conventional process, for example from the corresponding acid and base or by salt interchange.

Examples of suitable pharmaceutically acceptable salts include inorganic acid addition salts such as hydrochloride, hydrobromide, sulfate, phosphate, and nitrate; organic acid addition salts such as acetate, propionate, succinate, lactate, glycolate, malate, tartrate, citrate, maleate, fumarate, methansulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 plots a time kill of S. aureus by CBD over 24 days;

FIG. 2 plots the daily variability of time kill experiments of S. aureus by CBD over 24 days;

FIG. 3 plots the development of resistance to CBD by S. aureus over 24 days;

FIG. 4 plots the development of resistance to daptomycin by S. aureus over 24 days;

FIG. 5 plots the MIC distribution of S. aureus strains after treatment with Vancomycin, Daptomycin, Mupirocin, Clindamycin and Cannabidiol;

FIG. 6 plots the MIC distribution of S. aureus MRSA strains after treatment with Vancomycin, Daptomycin, Mupirocin, Clindamycin and Cannabidiol; and

FIG. 7 plots the MIC distribution of S. aureus MSSA strains after treatment with Vancomycin, Daptomycin, Mupirocin, Clindamycin and Cannabidiol.

FIG. 8 (a, b) is a graph of the results of an ex vivo pig skin model. Colony forming units (CFU) remaining on biopsy pig skin explants inoculated with S. aureus MRSA ATCC43300. Compositions containing CBD or mupirocin (solid colours) and compositions with no CBD (barred columns) were applied 2 h post-infection. At 1 h (a) or 24 h (b) later tissue was removed and CFU remaining determined (n=2-3, error bars show SEM; * denotes statistically significant deviation from Growth Control (p<0.05).

FIG. 9 is a graph of the results of an ex vivo pig skin model. Colony forming units (CFU) remaining on biopsy pig skin explants inoculated with S. aureus MRSA ATCC43300. Compositions containing CBD or mupirocin (solid colours) and compositions with no CBD (barred columns) were applied 2 h post-infection. At 1 h (a) or 24 h (b) later tissue was removed and CFU remaining determined (n=2-3, error bars show SEM; * denotes statistically significant deviation from Growth Control (p<0.05).

FIG. 10 is a graph of the results of an ex vivo pig skin model. Colony forming units (CFU) remaining on biopsy pig skin explants inoculated with S. aureus MRSA 329. Compositions containing CBD or mupirocin (solid colours) and compositions with no CBD (barred columns) were applied 2 h post-infection. At 1 h (a) or 24 h (b) later tissue was removed and CFU remaining determined (n=2-3, error bars show SEM; * denotes statistically significant deviation from Growth Control (p<0.05).

FIG. 11 is a graph of the results of an ex vivo pig skin model. Colony forming units (CFU) remaining on biopsy pig skin explants inoculated with S. aureus MRSA 993. Compositions containing CBD or mupirocin (solid colours) and compositions with no CBD (barred columns) were applied 2 h post-infection. At 1 h (a) or 24 h (b) later tissue was removed and CFU remaining determined (n=2-3, error bars show SEM; * denotes statistically significant deviation from Growth Control (p<0.05).

FIG. 12 is a graph of the results of an ex vivo pig skin model. Colony forming units (CFU) remaining on biopsy pig skin explants inoculated with S. aureus MRSA 815. Compositions containing CBD or mupirocin (solid colours) and compositions with no CBD (barred columns) were applied 2 h post-infection. At 1 h (a) or 24 h (b) later tissue was removed and CFU remaining determined (n=2-3, error bars show SEM; * denotes statistically significant deviation from Growth Control (p<0.05).

FIG. 13 is a graph of the irritation effects of the CBD-containing compositions or the associated vehicle, PBS, 10% Tween-20 in distilled water or 1% Triton in distilled water.

FIG. 14 is a graph of the results of the ex vivo pig skin model testing 5%, 10%, 15% or 20% CBD compositions against biopsy pig skin explants inoculated with S. aureus MRSA ATCC43300.

FIG. 15 is a graph of the results of the ex vivo pig skin model testing 5%, 10%, 15% or 20% CBD compositions against biopsy pig skin explants inoculated with S. aureus MRSA ATCC43300.

### EXAMPLES

### Example 1

This experiment was done to evaluate the ability of Cannabidiol (CBD) to disrupt Staphylococcus aureus MRSA ATCC 43300 biofilm formation. CBD was supplied by Dr Michael Thurn of Botanix Pharmaceuticals Ltd.

### Methods

Compound preparation

The collaborator supplied sample as dry material. A stock solution at 10 mg/mL in neat DMSO (11.2 mg in 1.12 mL of DMSO) was prepared. The highest concentration tested in the assay was 128 μg/mL and 2% DMSO as a final concentration using 1/20 dilution to achieve these concentrations.

### Biofilm Formation

Bacteria (Staphylococcus aureus, ATCC 43300; MRSA) was cultured on Tryptic Soy Broth (TSB, BD, Cat. No. 211825) at 37° C. overnight, then it was diluted 1:100 in fresh TSB supplemented with 5% glucose. 100 μL were added across the 96-well of polystyrene (PS) (Corning; Cat. No. 3370) plate, leaving row H as media Control. Plates were incubated at 37° C. for 48 h to generate the biofilm. The plates were prepared in duplicate.

### Biofilm Minimum Inhibitory Concentration (Biofilm MIC)

The antibiotic controls and CBD were serially diluted in TSB with 5% glucose two-fold across the wells of polystyrene (PS) 96-well plates (Corning; Cat. No. 3370), plated in duplicate. All plates had flat bottom wells and were covered with low-evaporation lids.

48 h after incubation, bacteria plates were carefully washed three times with 200 μL/well of saline solution (0.9% NaCl, Baxter Healthcare; Cat. No. AHF7124) using manual pipette to remove the planktonic cells but leave the biofilm adhered to the plate wells. Then, 100 μL of diluted controls and CBD were transferred into the washed plates containing the biofilm. Then, these plates were incubated at 37° C. for 24 h.

Next day, plates were washed three times with saline solution, then fixed with 100 μL/well of 99% methanol for 15 minutes. Once the biofilm was fixed, 100 μL/well 0.1% Crystal Violet Stain (Sigma; Cat No. C0775-25G) was added for 20 minutes and used as indicator of biofilm formation, followed by three times washing and dry well. To dissolve the crystal violet, 150 μL/well of methanol was added to allow for biofilm MIC analysis.

### Biofilm MIC Detection and Analysis

The biofilm formation was determined by optical density read at 590 nm (OD590). The percentage of biofilm formation was evaluated comparing the average, standard deviation and percentage of confidence of the media control (Row H) against the rest of the plate.

Inhibition of biofilm growth was determined as the lowest concentration at which OD590 demonstrated ≥70% growth inhibition compared to the growth control. Analysis was performed using Microsoft Excel.

TABLE 1
Tested Compound

| | | | | | Maximum | Minimum |
|---|---|---|---|---|---|---|
| | | Supplied | Stock | | test | test |
| | Sample | dry material | concentration | | concentration | concentration |
| MCC | name | (g) | (mg/mL) | Solvent | (μg/mL) | (μg/mL) |
| MCC_009427 | Cannabidiol | 5 | 10 | DMSO | 128 | 0.03 |
| | (CBD) | | | | | |

TABLE 2
Control Compounds

| | | Stock | | Target |
|---|---|---|---|---|
| | Compound | concentration | | organism |
| MCC | name | (mg/mL) | Source | class |
| MCC_000095 | Vancomycin | 0.64 | Sigma 861987 | Gram+ |
| | (HCl) | | | |
| MCC_000561 | Daptomycin | 1.28 | Molekula | Gram+ |
| | | | 64342447 | |
| MCC_000191 | Trimethoprim | 1.28 | Sigma T7883 | Gram+/− |
| MCC_009395 | Mupirocin | 0.64 | Glentham | Gram+ |
| | | | GA2184 | |
| MCC_008132 | Clindamycin | 0.64 | Glentham | Gram+ |
| | hydrochloride | | GA5034 | |
| | monohydrate | | | |

### Results

The results display two biological replicates, with technical replicates (total n=4).

TABLE 3
Broth MIC values
Staphylococcus aureus
ATCC 43300

| | | | TSB + 5% |
|---|---|---|---|
| | | CAMHB | Glucose |
| Compound ID | Compound name | Broth MIC (μg/mL) | |
| MCC_000095 | Vancomycin | 0.5 | 1 |
| MCC_000561 | Daptomycin | 0.5/1 | 32 |
| MCC_000191 | Trimethoprim | 1 | 4 |
| MCC_009395 | Mupirocin | 0.25 | 0.25 |
| MCC_008132 | Clindamycin* | >64 | >64 |
| MCC_009427 | Cannabidiol | 1 | 0.5 |

*note
clidamycin inactive vs this strain of MRSA

TABLE 4
Biofilm MIC values
Staphylococcus aureus
ATCC 43300

| | | Biofilm MIC | | Biofilm MIC | |
|---|---|---|---|---|---|
| | | (TSB + 5% Gluc) | | (TSB + 5% Gluc) | |
| | Compound | 07/112/18 | | 14/112/18 | |
| Compound ID | name | MIC (μg/mL) | | | |
| MCC_000095 | Vancomycin | 4 | 4 | 4 | 4 |
| MCC_000561 | Daptomycin | 32 | 16 | 16 | 16 |
| MCC_000191 | Trimethoprim | 8 | 8 | 16 | >64 |
| MCC_009395 | Mupirocin | 0.25 | 0.125 | 0.25 | 0.25 |
| MCC_008132 | Clindamycin | >64 | >64 | >64 | >64 |
| MCC_009427 | Cannabidiol | 4 | 4 | 2 | 2 |

CBD was capable of inhibiting up to 75% of 48 h biofilm formation at 2 and 4 μg/mL. The cannabidiol biofilm MIC was approximately four-fold higher (1-2 μg/mL) than its standard vegetative cell MIC (0.5-1 μg/mL) against the same strain of MRSA.

### Example 3

Antibacterial Time Kill Assay Staphylococcus aureus MRSA

Time-kill assay specifies a better descriptive assessment of cell killing (at a specific time) when compared to the single endpoint broth microdilution (MIC) assay. The assay determines the rate and the extent of antibacterial activity within a certain time period, and may also provide information on the possible in vivo activity of the antibacterial agents under study. This experiment was done to estimate how long it takes to Cannabidiol (CBD) to show antimicrobial activity against Staphylococcus aureus MRSA ATCC 43300. CBD was supplied by Dr Michael Thurn of Botanix Pharmaceuticals Ltd.

The time-kill method is based on CLSI guideline M26-A (NCCLS, 1999).

### Methods

### Compound Preparation

The collaborator supplied sample as dry material. A stock solution was prepared at 10 mg/mL in neat DMSO (11.2 mg in 1.12 mL of DMSO). The highest concentration tested in the assay was 64 μg/mL and 2% DMSO as a final concentration using 1/20 dilution to achieve these concentrations.

### Plate Assay Preparation

Time kill plates: CBD was plate across all the rows and serially diluted in Cation-adjusted Mueller Hinton Broth (CaMHB; BD, Cat. No. 212322) two-fold across the wells of polystyrene (PS) 96-well plates (Corning; Cat. No. 3370), plated in duplicate. Each row were taken as a time point, where row A, 0 h; row B, 1 h; row C, 2 h; row D, 3 h; row E, 4 h; row F, 6 h and row G, 24 h.

Also, control plates were made. CBD and standard antibiotics were serially diluted in Cation-adjusted Mueller Hinton Broth (CaMHB; BD, Cat. No. 212322) two-fold across the wells of polystyrene (PS) 96-well plates (Corning; Cat. No. 3370), plated in duplicate.

### Time Kill

The tested bacteria was Staphylococcus aureus ATCC 43300 MRSA (ID GP_020:02).

Charcoal plate PS 96-well plates: 50 μL of sterile activated charcoal suspension (25 mg/ml) were added into row A. 90 μL of 0.9% sterile saline were added to subsequent rows.

Bacteria (Table 2.6) was cultured in CaMHB at 37° C. overnight, then diluted 40-fold and incubated at 37° C. for a further 2-3 h. The resultant mid-log phase cultures were diluted in CaMHB and added to each well of the control and time kill 96-well plates to give a final cell density of 5×10⁵ CFU/mL, and a final compound concentration range of 0.03-64 μg/mL. The plates were covered and incubated at 37° C. for 24 h.

At selected time-points (0, 1, 2, 3, 4, 6 and 24 h), 50 μL of culture per-well was transferred from the time kill plate into the first row of charcoal plate (containing the charcoal suspension) to neutralise the compound. After mixing well, 10 μL were transferred from row A to row B to give a 1:10 dilution, this step was repeated until 1:10'000 (row E). Aliquots of each dilution was spotted in duplicate onto Tryptic soy agar (TSA; BD, Cat No. 236950) and incubated overnight at 37° C.

### MIC Detection and Analysis

MICs and the time kill results were determined visually at 24 hr incubation. The MIC was defined as the lowest concentration with which no growth was visible after incubation. The time kill was defined with growth/no growth of the colonies in each spot.

TABLE 5
Tested Compound

| | | Supplied | Stock | | Max test | Min |
|---|---|---|---|---|---|---|
| | Sample | dry material | con | | conc | test conc |
| MCC | name | (g) | (mg/mL) | Solvent | (μg/mL) | (μg/mL) |
| MCC_009427 | Cannabidiol | 5 | 10 | DMSO | 128 | 0.03 |
| AMRI supply | (CBD) | | | | | |

TABLE 6
Control Compounds

| | | | Stock | | Target |
|---|---|---|---|---|---|
| | | | conc | | organism |
| MCC | Compound name | MW | (mg/mL) | Source | class |
| MCC_000095 | Vancomycin (HCl) | 1485.71 | 0.64 | Sigma 861987 | Gram+ |
| MCC_000561 | Daptomycin | 1,619.701 | 1.28 | Molekula | Gram+ |
| | | | | 64342447 | |
| MCC_000191 | Trimethoprim | 290.32 | 1.28 | Sigma T7883 | Gram+/− |
| MCC_009395 | Mupirocin | 500.62 | 0.64 | Glentham | Gram+ |
| | | | | GA2184 | |
| MCC_008132 | Clindamycin | 504.96 | 0.64 | Glentham | Gram+ |
| | hydrochloride | | | GA5034 | |
| | monohydrate | | | | |

### Results

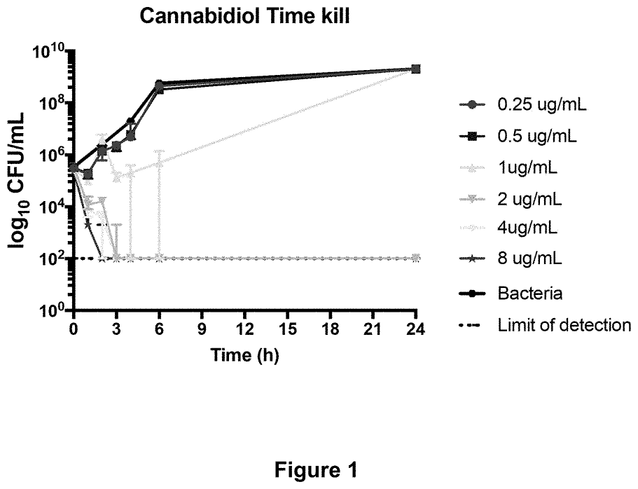

CBD time kill was tested two concentrations above and below previous MIC data (1-2 μg/mL). CBD control MIC of the day was 2 μg/mL. Tested concentrations over or equal to the MIC value showed to be bactericidal after 3 hour treatment (FIG. 1).

### Example 4

Forced Evolution of Resistant in Staphylococcus aureus MRSA

This experiment was done to assess the development of resistance over 20 days of growth of Staphylococcus aureus (ATCC 43300) in the presence of sub-inhibitory concentrations of Cannabidiol (CBD) and daptomycin (used as a positive control), conducted in parallel in eight replicates.

### Methods

### Compound Preparation

The collaborator supplied sample as dry material. A stock solution at 10 mg/mL in neat DMSO was prepared.

### Viability Testing

The tested bacteria was Staphylococcus aureus ATCC 43300 MRSA (ID GP_020:02).

The mid log Staphylococcus aureus (ATCC 43300) growth culture was serially diluted and plated on a solid Tryptic Soy Agar (TSA) plates in duplicates and incubated at 37° C. overnight to determine viable colony count.

CBD 320 μg/mL stock was diluted to 5, 4, 3, 2, 1.5, 1, 0.75, 0.5, 0.375 and 0.25 μg/mL in Cation-adjusted Mueller Hinton Broth (CaMHB; BD, Cat. No. 212322) 100 μL were plated from well 1 to 10 across the wells of polystyrene (PS) 96-well plates (Corning; Cat. No. 3370). Staphylococcus aureus (ATCC 43300) was cultured in CaMHB at 37° C. overnight, then diluted 40-fold and incubated at 37° C. for a further 2-3 h. The resultant mid-log phase cultures were diluted in CaMHB and 100 pL added to each well of the compound-containing 96-well plates to give a final cell density of 5×10⁵ CFU/mL. The plate was covered and incubated at 37° C. for 20 h.

Note: CBD will have 8 replicates.

MICs were determined visually at 24 h incubation and the MIC was defined as the lowest concentration with which no growth was visible after incubation.

### Bacteria Preparation:

Plate well with the highest drug concentration that permitted growth was then diluted. Despite plates were read by eye, reading at OD600 on the Epoch microplate spectrophotometer was used to adjust growth density of each well (it was approximately 1:1000). Then, 100 μL of the bacteria diluted was added to the new MIC plate. The OD₆₀₀ was used to calculate the dilution of cells to a density of 10₆ CFU/mL. Bacteria were diluted in CaMHB and 100 μL was added to each well of the next replicate passage. The final well volume was 200 μL with a cell density of 5×10₅ CFU/mL. Each replicate (row) was assess as different strain, for this reason the dilution was done for each replicate.

Once prepared, the plate was covered and incubated at 37° C. overnight. Plate reading, compound preparation and bacterial preparation were repeated from Day 2 to Day 20.

### Compound Preparation:

Depending on the MIC of the day before, CBD tested concentrations were established.

Depending on the MIC of the day before, CBD and daptomycin tested concentrations were established to ensure at least three concentrations above, and three concentrations below MIC, based on the previous MIC results. Compounds were prepared in Protein LoBind Eppendorf 1.5 mL safelock tubes, diluting 320 μg/mL stock in DMSO in CaMHB to achieve two-fold the desired testing concentrations. The 100 μL of the selected concentration were added to each well (See FIG. 1). Once the plate had 100 μL of bacteria and 100 μL of compound. It was incubated at 37° C. overnight. Next day the same procedure was repeated.

### Drug Free Passages

Following 20 days of passaging in the presence of CBD and daptomycin, each replicate was passaged for 4 days in drug-free media to assess the stability of any induced resistance.

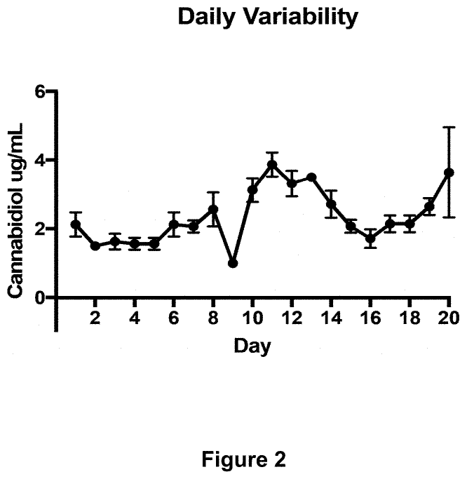

Day 20 plate was read and the same bacterial preparation methodology was followed. Same concentrations used in day 20 for CBD were used for the 4 days drug free passages. Daptomycin 320 μg/mL stock was diluted to 16, 8, 5, 4, 2.5, 2, 1.25, 1, 0.75 and 0.5. These concentrations were used for the 4 days drug free passages. Column 11 was used as the drug-free passage well, and column 12 as a negative growth control with 200 μL uninoculated media in each well. Diluted bacteria were added to the plate, one replicate per row, 100 μL per well. The final well volume was 200 μL with a cell density of 5×105 CFU/mL in columns 1-11, and CBD concentration range from 16-0.03 μg/mL in columns 1-10 (FIG. 2).

Subsequent drug-free passage plates were prepared in the same manner, except each replicate bacteria was passaged from column 11, the drug-free growth control well.

TABLE 7
Tested Compound

| | | | | | Maximum | Minimum |
|---|---|---|---|---|---|---|
| | | Supplied | Stock | | test | test |
| | Sample | dry material | concentration | | concentration | concentration |
| MCC | name | (g) | (mg/mL) | Solvent | (μg/mL) | (μg/mL) |
| MCC_009427 | Cannabidiol | 5 | 10 | DMSO | 128 | 0.03 |
| AMRI supply | (CBD) | | | | | |
| Batch ref | | | | | | |
| R0030516 | | | | | | |
| RM342K.0706 | | | | | | |

TABLE 8
Control Compounds

| | | | Stock | | Target |
|---|---|---|---|---|---|
| | Compound | | concentration | | organism |
| MCC | name | MW | (mg/mL) | Source | class |
| MCC_000095 | Vancomycin | 1485.71 | 0.64 | Sigma 861987 | Gram+ |
| | (HCl) | | | | |
| MCC_008136 | Erythromycin | 733.93 | 0.64 | Avistron | Gram+ |
| | | | | AE22796 | |
| MCC_000236 | Oxacillin sodium | 401.43 | 0.64 | Sigma O1002- | Gram+ |
| | salt hydrate | | | 1G | |
| MCC_000167 | Tetracycline | 480.90 | 0.64 | Sigma T7660- | Gram+ |
| | hydrochloride | | | 5G | |
| MCC_009395 | Mupirocin | 500.62 | 0.64 | Glentham | Gram+ |
| | | | | GA2184 | |
| MCC_008132 | Clindamycin | 504.96 | 0.64 | Glentham | Gram+ |
| | hydrochloride | | | GA5034 | |
| | monohydrate | | | | |

### Drug-free Passaging Control & QC Plate

Alongside the test plate, a culture of S. aureus was passaged for 24 days without CBD, to establish a baseline for non-selective mutations in the growth conditions described.

In a PS 96-well plate control compounds (see Control compounds) were serially, two-fold diluted in CaMHB across the rows of columns 1-12 to give a final volume of 50 μL of 2× the desired test concentration. Six wells were used as a positive growth controls, and six as negative growth controls with uninoculated media.

On day one, mid-log phase S. aureus was diluted in CaMHB to 106 CFU/mL, and 50 μL was added to each well (except negative growth control wells), to give a final volume of 100 μL and concentration of 5×105 CFU/mL.

Subsequent passages were inoculated from well H7. The bacterial growth in H7 was resuspended by pipetting, then plates were read for optical density by spectrophotometer (Biotek Epoch) at 600 nm (OD600). The OD600 was used to calculate the dilution of cells to a density of 106 CFU/mL. Bacteria were diluted in CaMHB and 50 μL was added to each well of the next passage. The final well volume was 100 μL with a cell density of 5×105 CFU/mL.

### Results

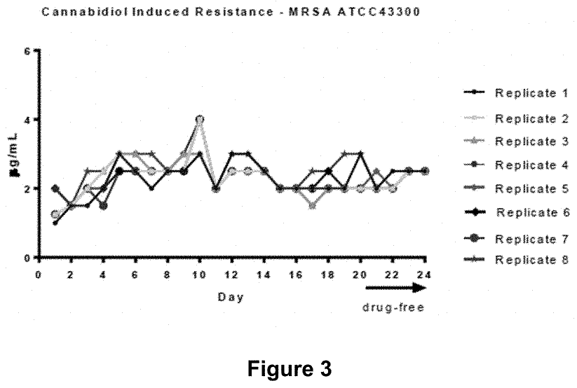

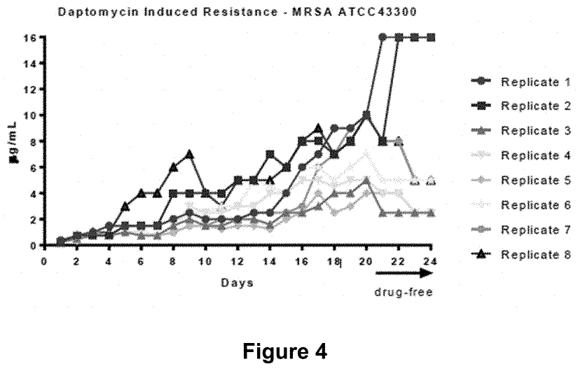

Through the 20 days of assay, CBD generally showed a constant activity between 2 to 4 μg/mL across most of the replicates (FIGS. 3 and 4). However, replicate 1 had a drastic increase of activity from 3.5 μg/mL to >7 μg/mL at day 13 (the highest concentration tested that day), and the MIC exceeded the highest concentration tested on subsequent days (up to >128 μg/mL) by day 18 (FIG. 2). This replicate is currently under 16S and purity studies to confirm that it is not a contaminant. The results for this replicate after day 7 have been excluded from FIGS. 3 and 4. During the course of the experiment technical difficulties on the 17th day meant the assay plates were stored at 4° C. for 24 h, with the assay then continued without disruption. There was also a consistent drop in measured MIC on Day 9 across all replicates to 1 μg/mL, with no obvious explanation.

Following the 20 days induction the 8 replicates were subcultured for an additional 5 days of drug free passages to test for stability of any induced resistance. The final MIC were generally within the variability range of the samples, however replicates 2 and 8 did consistently show elevated MIC (6-16 μg/mL) on Days 20 and 21.

### Example 5

### Minimum Inhibitory Concentration in Presence of 50% Human Serum

This experiment was done assess the activity of Cannabidiol (CBD) for antimicrobial activity against three strains of Staphylococcus aureus in the presence of 50% human serum.

### Methods

### Compound Preparation

The collaborator supplied sample as dry material. A stock solution at 10 mg/mL in neat DMSO was prepared. The highest concentration tested in the assay was 1.28 mg/mL and 2% DMSO as a final concentration using 1/20 dilution to achieve these concentrations.

### Minimum Inhibitory Concentration (MIC) Micro-broth Dilution Assay

The compounds were serially diluted in mixture of 50% of human serum (Sigma; Cat. No. H3667-100ML) along with 50% Cation-adjusted Mueller Hinton Broth (CaMHB; BD, Cat. No. 212322) two-fold across the wells of polystyrene (PS) 96-well plates (Corning; Cat. No. 3370), plated in duplicate. All plates had flat bottom wells and were covered with low-evaporation lids.

Staphylococcus aureus strains were cultured in CaMHB at 37° C. overnight, then diluted 40-fold and incubated at 37° C. for a further 2-3 h. The resultant mid-log phase cultures were diluted in CaMHB and added to each well of the compound-containing 96-well plates to give a final cell density of 5×10⁵ CFU/mL, and a final compound concentration range of 0.03-64 μg/mL. The plates were covered and incubated at 37° C. for 20 h.

### MIC Detection and Analysis

The MIC was defined as the lowest concentration with which no growth was visible after incubation. MIC was determined by visual inspection only.

TABLE 9
Tested Compound

| | | Supplied | Stock | | Max test | Min test |
|---|---|---|---|---|---|---|
| | Sample | dry material | conc | | conc | conc |
| MCC | name | (g) | (mg/mL) | Solvent | (μg/mL) | (μg/mL) |
| MCC_009427 | Cannabidiol | 5 | 10 | DMSO | 64 | 0.03 |
| | (CBD) | | | | | |

TABLE 10
Control Compounds

| | | | Stock | | Target |
|---|---|---|---|---|---|
| | Compound | | concentration | | organism |
| MCC | name | MW | (mg/mL) | Source | class |
| MCC_000095 | Vancomycin | 1485.71 | 1.28 | Sigma | Gram+ |
| | (HCl) | | | 861987 | |
| MCC_000561 | Daptomycin | 1,619.701 | 1.28 | Molekula | Gram+ |
| | | | | 64342447 | |
| MCC_000191 | Trimethoprim | 290.32 | 1.28 | Sigma T7883 | Gram+ |
| MCC_008132 | Clindamycin | 504.96 | 1.28 | Glentham | Gram+ |
| | hydrochloride | | | GA5034 | |
| | monohydrate | | | | |
| MCC_009395 | Mupirocin | 500.62 | 1.28 | Glentham | Gram+ |
| | | | | GA2184 | |

TABLE 11
Tested Bacteria

| ID | Species | Strain | Description |
|---|---|---|---|
| GP_020:02 | Staphylococcus | ATCC 43300 | MRSA |
| | aureus | | |
| GP_035:01 | Staphylococcus | ATCC 700699, | MRSA, |
| | aureus | NRS1 | VISA |
| GP_064:01 | Staphylococcus | NARSA, VRS1 | VRSA |
| | aureus | | |

### Results

For bacteria, two technical duplicates.

TABLE 12
Summary of Result

| | | | | GP_064 |
|---|---|---|---|---|
| | | GP_020 | GP_035 | S. aureus |
| | | S. aureus | S. aureu | NARSA, |
| | Compound | MRSA | NRS60 | RS1 |
| Compound ID | Name | MIC (μg/mL) | | |
| MCC_000095_002 | Vancomycin | 1 | 4/16 | >64 |
| MCC_000561_002 | Daptomycin | 4 | 16 | >64 |
| MCC_000191_002 | Trimethoprim | 4 | 8 | >64 |
| MCC_009395_001 | Mupirocin | 4/8 | 4/8 | >64 |
| MCC_008132_001 | Clindamycin | >64 | >64 | >64 |
| | hydrochloride | | | |
| MCC_009427_002 | Cannabidiol | >64 | >64 | >64 |

All control antibiotics gave inhibitory values within the expected ranges. CBD was inactive against all tested strains when human serum was added to the assay medium, consistent with high levels of protein binding (e.g. >97% assuming 3% free responsible for activity).

Below is a summary of the Minimum Inhibitory Concentration (MIC) range for each compound. The experiment was performed with two technical duplicates (n=2). Where the duplicate readings are the same a single value is displayed. Two values are displayed where the duplicates differed.

TABLE 13
Summary of Results

| | | | | GP_064 | |
|---|---|---|---|---|---|
| | | | GP_035 | S. aureus | |
| | | S. aureus | S. aureus | NARSA, | S. aureus |
| Compound | Compound | MRSA | NRS60 | VRS1 | MRSA |
| ID | Name | MIC (ug/mL) | | | |
| MCC_ | Cannabidiol | no serum* | 1* | 2* | 2* |
| 009427_002 | | +50% | >64 | >64 | >64 |
| | | human | | | |
| | | serum | | | |

*from report 99962_002

### Example 6

Minimum Inhibitory Concentration Assays MIC90 vs S. aureus

This experiment was done to assess the antimicrobial activity of Cannabidiol (CBD) against 132 strains of Staphylococcus aureus (106 MRSA and 26 MSSA strains).

### Methods

### Compound Preparation

The collaborator supplied the sample as dry material. A stock solution at 10 mg/mL in neat DMSO was prepared. The highest concentration tested in the assay was 32 μg/mL. 5% DMSO was the final concentration using 1/10 dilution to achieve these concentrations.

### Bacterial Minimum Inhibitory Concentration (MIC) Micro-broth Dilution Assay

The compounds were serially diluted in sterile water two-fold across a polypropylene (PP) 96-deep well plate (Fisher Biotec; Cat No. AX-P-DW-20-C-S) and 10 μL were stamped into polystyrene (PS) 96-well plates (Corning; Cat. No. 3370).

Staphylococcus aureus were cultured in Cation-adjusted Mueller Hinton Broth (CaMHB; BD, Cat. No. 212322) at 37° C. overnight, then diluted 40-fold and incubated at 37° C. for a further 2-3 h. The resultant mid-log phase cultures were diluted in CaMHB and added to each well of the compound-containing 96-well plate to give a final cell density of 5×10⁵ CFU/mL, and a final compound concentration range of 0.03-64 μg/mL. The plates were covered and incubated at 37° C. for 20 h.

### Bacterial MIC Detection and Analysis

Optical density was read at 600 nm (OD600) using Tecan M1000 Pro Spectrophotometer. MIC was determined as the lowest concentration at which 95% growth inhibition was observed. Dr Johannes Zuegg wrote script algorithms using Pipeline Pilot to automatically analyse the data set.

The quality control (QC) of the assays was determined by Z′-Factor, calculated from the Negative (media only) and Positive Controls (bacterial without inhibitor), and the Standards. Plates with a Z′-Factor of ≥0.25 and Standards active at the highest and inactive at the lowest concentration, were accepted for further data analysis.

MIC 90 and 50 analysis was performed using Microsoft Excel.

TABLE 14
Tested Compound

| | | Supplied | Stock | | Max test | Min test |
|---|---|---|---|---|---|---|
| | Sample | dry material | conc | | conc | conc |
| MCC | name | (g) | (μg/mL) | Solvent | (μg/mL) | (μg/mL) |
| MCC_009427 | Cannabidiol | 5 | 320 | DMSO | 32 | 0.015 |
| Norenco supply | (CBD) | | | | | |
| Batch ref | | | | | | |
| 0030516K.0706 | | | | | | |

TABLE 15
Control Compounds

| | | | Stock | | Target |
|---|---|---|---|---|---|
| | Compound | | cone | | organism |
| MCC | name | MW | (μg/mL) | Source | class |
| MCC_000095 | Vancomycin | 1485.71 | 640 | Sigma | Gram + |
| | (HCL) | | | 861987 | |
| MCC_000561 | Daptomycin | 1,619.701 | 640 | Molekula | Gram + |
| | | | | 64342447 | |
| MCC_009395 | Mupirocin | 500.62 | 640 | Glentham | Gram + |
| | | | | GA2184 | |
| MCC_008132 | Clindamycin | 504.96 | 640 | Glentham | Gram + |
| | hydrochloride | | | GA5034 | |
| | monohydrate | | | | |

TABLE 16
Tested Staphylococcus aureus strains

| ID | Strain | Description |
|---|---|---|
| GP_001 | ATCC 25923 | Control |
| GP_003 | CI Paterson 404556145 | Clinical Isolate |
| GP_004 | CI Paterson 405575036 | Clinical Isolate |
| GP_005 | CI Paterson 406626061 | Clinical Isolate |
| GP_006 | CI Paterson 422940878 | Clinical Isolate |
| GP_007 | CI Paterson 414149225 | Clinical Isolate |
| GP_008 | CI Paterson 405573757 | Clinical Isolate |
| GP_010 | CI Paterson 405574456 | Clinical Isolate; Resistant |
| GP_020 | ATCC 43300 | Resistant |
| GP_021 | ATCC 33591 | Resistant |
| GP_022 | ATCC 29213 | Control |
| GP_028 | NRS 119 | Resistant |
| GP_029 | NRS2; ATCC 700698 | Resistant |
| GP_030 | NRS 17 | Resistant |
| GP_031 | NRS 18 | Resistant |
| GP_032 | NRS 19 | Resistant |
| GP_034 | NRS 384 | Resistant |
| GP_035 | NRS 1; Mu50; ATCC 700699 | Resistant |
| GP_036 | CI Paterson 581101692:1 | Clinical Isolate; Resistant |
| GP_037 | CI Paterson 581101692:2 | Clinical Isolate; Resistant |
| GP_038 | CI Paterson 581101692:3 | Clinical Isolate; Resistant |
| GP_047 | 50316-0509 | Clinical Isolate; Resistant |
| GP_049 | 51418-7407 | Clinical Isolate; Resistant |
| GP_050 | 49496-1320 | Clinical Isolate; Resistant |
| GP_062 | VRS3b | Resistant |
| GP_063 | VRS4 | Resistant |
| GP_064 | VRS1 | Resistant |
| GP_065 | VRS10 | Resistant |
| GP_097 | M30538 | Clinical Isolate |
| GP_098 | M31394 | Clinical Isolate |
| GP_099 | M31634 | Clinical Isolate |
| GP_100 | M31907 | Clinical Isolate |
| GP_101 | M32158 | Clinical Isolate |
| GP_102 | M32158 | Clinical Isolate |
| GP_103 | M34027 | Clinical Isolate |
| GP_104 | M34575 | Clinical Isolate |
| GP_105 | M34591 | Clinical Isolate |
| GP_106 | M34593 | Clinical Isolate |
| GP_108 | M35252 | Clinical Isolate |
| GP_109 | M35254 | Clinical Isolate |
| GP_110 | M35255 | Clinical Isolate |
| GP_111 | M35264 | Clinical Isolate |
| GP_112 | M35268 | Clinical Isolate |
| GP_113 | M35491 | Clinical Isolate |
| GP_114 | M35953 | Clinical Isolate |
| GP_115 | M36523 | Clinical Isolate |
| GP_116 | M37410 | Clinical Isolate |
| GP_117 | M33376 | Clinical Isolate; Resistant |
| GP_118 | M35249 | Clinical Isolate; Resistant |
| GP_119 | M38184 | Clinical Isolate; Resistant |
| GP_120 | M31414 | Clinical Isolate; Resistant |
| GP_121 | M38509 | Clinical Isolate; Resistant |
| GP_122 | M39864 | Clinical Isolate; Resistant |
| GP_123 | M40725 | Clinical Isolate; Resistant |
| GP_124 | M45447 | Clinical Isolate; Resistant |
| GP_125 | M48439 | Clinical Isolate; Resistant |
| GP_126 | M49406 | Clinical Isolate; Resistant |
| GP_127 | M51977 | Clinical Isolate; Resistant |
| GP_128 | M52817 | Clinical Isolate; Resistant |
| GP_129 | M54307 | Clinical Isolate; Resistant |
| GP_130 | M53519 | Clinical Isolate; Resistant |
| GP_131 | M55707 | Clinical Isolate; Resistant |
| GP_132 | M56123 | Clinical Isolate; Resistant |
| GP_133 | M48662 | Clinical Isolate; Resistant |
| GP_134 | M49378 | Clinical Isolate; Resistant |
| GP_135 | M49411 | Clinical Isolate; Resistant |
| GP_136 | M56924 | Clinical Isolate; Resistant |
| GP_137 | M57543 | Clinical Isolate; Resistant |
| GP_138 | M57544 | Clinical Isolate; Resistant |
| GP_139 | M59014 | Clinical Isolate; Resistant |
| GP_140 | M60609 | Clinical Isolate; Resistant |
| GP_141 | M76385 | Clinical Isolate; Resistant |
| GP_142 | M61448 | Clinical Isolate; Resistant |
| GP_143 | M63450 | Clinical Isolate; Resistant |
| GP_144 | M74145 | Clinical Isolate; Resistant |
| GP_145 | M74568 | Clinical Isolate; Resistant |
| GP_146 | M75365 | Clinical Isolate; Resistant |
| GP_147 | M76558 | Clinical Isolate; Resistant |
| GP_148 | M77399 | Clinical Isolate; Resistant |
| GP_149 | M78036 | Clinical Isolate; Resistant |
| GP_150 | M78540 | Clinical Isolate; Resistant |
| GP_151 | M81239 | Clinical Isolate; Resistant |
| GP_152 | M81986 | Clinical Isolate; Resistant |
| GP_153 | M82747 | Clinical Isolate; Resistant |
| GP_154 | M85049 | Clinical Isolate; Resistant |
| GP_155 | M85511 | Clinical Isolate; Resistant |
| GP_156 | M78411 | Clinical Isolate; Resistant |
| GP_157 | M87512 | Clinical Isolate; Resistant |
| GP_158 | M90736 | Clinical Isolate; Resistant |
| GP_159 | M89569 | Clinical Isolate; Resistant |
| GP_160 | M88418 | Clinical Isolate; Resistant |
| GP_161 | M88210 | Clinical Isolate; Resistant |
| GP_162 | M97784 | Clinical Isolate; Resistant |
| GP_163 | M97166 | Clinical Isolate; Resistant |
| GP_164 | M96912 | Clinical Isolate; Resistant |
| GP_165 | M234215 | Clinical Isolate; Resistant |
| GP_166 | M121493 | Clinical Isolate; Resistant |
| GP_167 | M69739 | Clinical Isolate; Resistant |
| GP_168 | M69740 | Clinical Isolate; Resistant |
| GP_169 | M70241 | Clinical Isolate; Resistant |
| GP_170 | M70964 | Clinical Isolate; Resistant |
| GP_171 | M71121 | Clinical Isolate; Resistant |
| GP_172 | M71122 | Clinical Isolate; Resistant |
| GP_173 | M72749 | Clinical Isolate; Resistant |
| GP_174 | M72760 | Clinical Isolate; Resistant |
| GP_175 | M73508 | Clinical Isolate; Mutant |
| GP_176 | M74801 | Clinical Isolate; Resistant |
| GP_177 | M74804 | Clinical Isolate; Resistant |
| GP_178 | M64647 | Clinical Isolate; Resistant |
| GP_179 | M65412 | Clinical Isolate; Resistant |
| GP_180 | M65412 | Clinical Isolate; Resistant |
| GP_181 | M66471 | Clinical Isolate; Resistant |
| GP_182 | M66723 | Clinical Isolate; Resistant |
| GP_183 | M67645 | Clinical Isolate; Resistant |
| GP_184 | M67826 | Clinical Isolate; Resistant |
| GP_185 | M67934 | Clinical Isolate; Resistant |
| GP_186 | M68334 | Clinical Isolate; Resistant |
| GP_187 | M69124 | Clinical Isolate; Resistant |
| GP_188 | M72169 | Clinical Isolate; Resistant |
| GP_189 | M72746 | Clinical Isolate; Resistant |
| GP_190 | M73705 | Clinical Isolate; Mutant |
| GP_191 | M75392 | Clinical Isolate; Resistant |
| GP_192 | M75683 | Clinical Isolate; Resistant |
| GP_193 | M75856 | Clinical Isolate; Resistant |
| GP_194 | M75899 | Clinical Isolate; Resistant |
| GP_195 | M76067 | Clinical Isolate; Resistant |
| GP_196 | M76386 | Clinical Isolate; Resistant |
| GP_221 | ATCC 43300 | Mutant Induced |
| | | (Daptomycin MRSA evolution) |
| GP_223 | ATCC 43300 | Mutant Induced |
| | | (Linezolid MRSA evolution) |
| GP_224 | ATCC 43300 | Mutant Induced |
| | | (Dalvamycin MRSA evolution) |
| GP_229 | ATCC 6538; FDA 209 | |
| GP_234 | ATCC 43300 | Mutant Induced |
| | | (CBD MRSA evolution) |

### Results

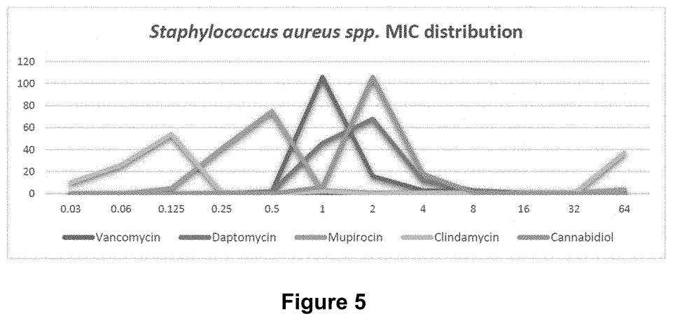

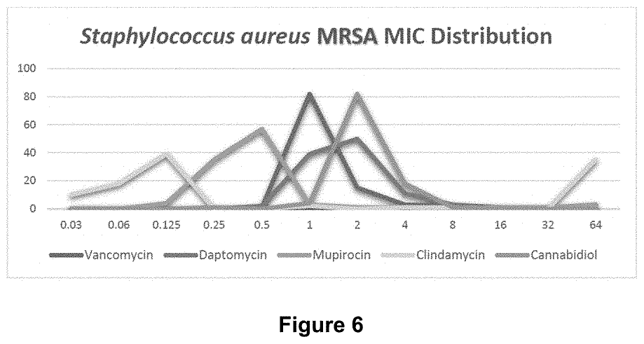

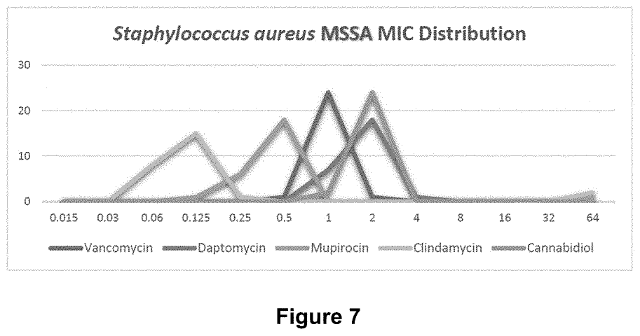

Out of the 132 strains, 37 were resistant to clindamycin, resulting in an MIC50 of 0.125 μg/mL changing to an MIC90 of 64 μg/mL. The other control antibiotics gave inhibitory values within the expected ranges. While several VISA/VRSA strains were resistant or highly resistant to vancomycin, there were not enough strains to substantially shift the MIC90. CBD showed a stable MIC between 2 to 4 μg/mL across the 132 strains tested. The assay was performed in two different days in duplicate (total n=4). See FIGS. 5-7.

TABLE 17
Summary of results

| | S. aureus spp. | | | S. aureus MSSA | | S. aureus MRSA | |
|---|---|---|---|---|---|---|---|
| | ALL (μg/mL) | | | (μg/mL) | | (μg/mL) | |
| | MIC 50 | MIC 90 | range | MIC 50 | MIC 90 | MIC 50 | MIC 90 |
| Vancomycin | 1 | 2 | 0.5-64 | 1 | 1 | 1 | 2 |
| Daptomycin | 2 | 4 | 0.5-16 | 2 | 2 | 2 | 4 |
| Mupirocin | 0.5 | 0.5 | 0.125-64 | 0.5 | 0.5 | 0.5 | 0.5 |
| Clindamycin | 0.125 | 64 | 0.03-64 | 0.125 | 0.1875 | 0.125 | 64 |
| Cannabidiol | 2 | 4 | 0.25-8 | 2 | 2 | 2 | 4 |

TABLE 18
Staphylococcus aureus spp. MIC distribution (μg/mL)
Staphylococcus aureus spp. MIC distribution (μg/mL)

| | 0.015 | 0.03 | 0.06 | 0.125 | 0.25 | 0.5 | 1 | 2 | 4 | 8 | 16 | 32 | 64 |
|---|---|---|---|---|---|---|---|---|---|---|---|---|---|
| Vanco | — | 0 | 0 | 0 | 0 | 2 | 106 | 16 | 3 | 2 | 0 | 0 | 3 |
| Dapto | — | 0 | 0 | 0 | 0 | 2 | 46 | 68 | 12 | 3 | 1 | 0 | 0 |
| Mupir | — | 0 | 0 | 5 | 41 | 75 | 3 | 1 | 1 | 0 | 1 | 1 | 4 |
| Clinda | — | 10 | 26 | 54 | 1 | 0 | 2 | 0 | 1 | 1 | 0 | 0 | 37 |
| CBD | 0 | 0 | 0 | 0 | 1 | 0 | 6 | 106 | 18 | 1 | 0 | 0 | — |

TABLE 19
Staphylococcus aureus MRSA MIC distribution
Staphylococcus aureus MRSA MIC distribution

| | 0.015 | 0.03 | 0.06 | 0.125 | 0.25 | 0.5 | 1 | 2 | 4 | 8 | 16 | 32 | 64 |
|---|---|---|---|---|---|---|---|---|---|---|---|---|---|
| Vanco | — | 0 | 0 | 0 | 0 | 1 | 82 | 15 | 3 | 2 | 0 | 0 | 3 |
| Dapto | — | 0 | 0 | 0 | 0 | 2 | 39 | 50 | 11 | 3 | 1 | 0 | 0 |
| Mupir | — | 0 | 0 | 4 | 35 | 57 | 3 | 1 | 1 | 0 | 1 | 1 | 3 |
| Clinda | — | 10 | 18 | 39 | 0 | 0 | 2 | 0 | 1 | 1 | 0 | 0 | 35 |
| CBD | 0 | 0 | 0 | 0 | 1 | 0 | 4 | 82 | 18 | 1 | 0 | 0 | — |

TABLE 20
Staphylococcus aureus MRSA MIC distribution
Staphylococcus aureus MRSA MIC distributio

| | 0.015 | 0.03 | 0.06 | 0.125 | 0.25 | 0.5 | 1 | 2 | 4 | 8 | 16 | 32 | 64 |
|---|---|---|---|---|---|---|---|---|---|---|---|---|---|
| Vanco | — | 0 | 0 | 0 | 0 | 1 | 24 | 1 | 0 | 0 | 0 | 0 | 0 |
| Dapto | — | 0 | 0 | 0 | 0 | 0 | 7 | 18 | 1 | 0 | 0 | 0 | 0 |
| Mupir | — | 0 | 0 | 1 | 6 | 18 | 0 | 0 | 0 | 0 | 0 | 0 | 1 |
| Clinda | — | 0 | 8 | 15 | 1 | 0 | 0 | 0 | 0 | 0 | 0 | 0 | 2 |
| CBD | 0 | 0 | 0 | 0 | 0 | 0 | 2 | 24 | 0 | 0 | 0 | 0 | — |

### Example 7

### Anaerobic Gram-positive Bacteria Minimum Inhibitory Concentration Assays

To assess the potential of Cannabidiol (CBD) for antimicrobial activity against common skin bacteria under anaerobic conditions.

### Methods

### Compound Preparation

The collaborator supplied sample as dry material. A stock solution at 10 mg/mL in neat DMSO was prepared. The highest concentration tested in the assay was 128 μg/mL and 2% DMSO as a final concentration using 1/20 dilution to achieve these concentrations.

### Minimum Inhibitory Concentration (MIC) Micro-broth Dilution Assay

All steps were performed in a COY type B anaerobic chamber with the anaerobic atmosphere controlled by the introduction of 10% CO2/5% H2 in N2CoA gas mix, catalyst Stak-Pak and O₂-H₂ gas analyser, with H2 levels kept at ˜2% for the duration of the assay. Brain Heart Infusion broth (BHI; OXOID CM1135B) media with 1% cysteine to further promote an anaerobic environment was used for this assay, and this broth was incubated in the anaerobic chamber for 24 h prior to use for reduction of oxygen.

CBD and control antibiotics were serially diluted in BHI, two-fold across the wells of 96-well of polystyrene (PS) 96-well plates (Corning; Cat. No. 3370). Plates were set up in duplicate for each strain.

All bacteria strains (Table 2.5) were cultured on Tryptic Soy agar (TSA, BD, Cat. No. 236950) at 37° C. for 72 h. A few colonies were taken from the agar plate and dissolved in BHI broth. The solution was then adjusted to OD₆₀₀ 0.5-0.7 and diluted down to a final cell density of 5×10⁵ CFU/mL, 100 μL were added to the test plate, giving a final CBD concentration range of 0.06-128 μg/mL. All the plates were covered and incubated at 37° C. for 48 h.

### MIC Detection and Analysis

The MIC was defined as the lowest concentration at which no growth was visible after incubation. MIC was determined by visual inspection only.

TABLE 21
Tested Compound

| | | | Stock | | Max test | Min test |
|---|---|---|---|---|---|---|
| | Sample | Supplied dry | conc | | conc | conc |
| MCC | name | material (g) | (mg/mL) | Solvent | (μg/mL) | (μg/mL) |
| MCC_009427 | Cannabidiol | 5 | 10 | DMSO | 128 | 0.03 |
| AMRI supply | (CBD) | | | | | |
| Batch ref | | | | | | |
| R0030516 | | | | | | |
| RM342K.0706 | | | | | | |

TABLE 22
Control Compounds

| | | | Stock | | |
|---|---|---|---|---|---|
| | | | cone | | Target |
| | Compound | | (mg/ | | organism |
| MCC | name | MW | mL) | Source | class |
| MCC_000095 | Vancomycin | 1485.71 | 0.64 | Sigma | Gram + |
| | (HCI) | | | 861987 | |
| MCC_008136 | Erythromycin | 733.93 | 0.64 | Avistron | Gram + |
| | | | | AE22796 | |
| MCC_000236 | Oxacillin sodium | 401.43 | 0.64 | Sigma | Gram + |
| | salt hydrate | | | O1002-1G | |
| MCC_000167 | Tetracycline | 480.90 | 0.64 | Sigma | Gram + |
| | hydrochloride | | | T7660-5G | |
| MCC_009395 | Mupirocin | 500.62 | 0.64 | Glentham | Gram + |
| | | | | GA2184 | |
| MCC_008132 | Clindamycin | 504.96 | 0.64 | Glentham | Gram + |
| | hydrochloride | | | GA5034 | |
| | monohydrate | | | | |

TABLE 23
Test Organisms

| ID | Species | Strain | Description |
|---|---|---|---|
| GP_020:02 | Staphylococcus aureus | ATCC 43300 | MRSA |
| GP_202:01 | Cutibacterium acnes (formerly | ATCC 6919 | Type strain |
| | Propionibacterium acnes) | | |
| GP_203:01 | Acidipropionibacterium | ATCC 25562 | Type strain |
| | acidipropionici | | |
| GP_204:01 | Cutibacterium granulosum | ATCC 25564 | Type strain |

### Results

For bacteria, two biological replicates, with technical replicates (total n=4). All control antibiotics gave inhibitory values within the expected ranges. The Cannabidiol (CBD) was active against all tested strains.

Below is a summary of the Minimum Inhibitory Concentration (MIC) range for each compound, determined in an anaerobic chamber in the absence of oxygen. The experiment was performed with two biological replicates of technical duplicates (n=4). Where the duplicate readings are the same a single value is displayed. Two values are displayed where the duplicates differed.

TABLE 24
Summary of Results

| | P. acnes | A. | C. | |
|---|---|---|---|---|
| | ATCC | acidipropionici | granulosum | S. aureus |
| Compound | 6919 | ATCC 25562 | ATCC 25564 | ATCC 43300 |
| Name | MIC (μg/mL) | | | |
| Vancomycin | 0.25 | 0.25/0.125 | 0.25 | 1/0.5 |
| Erythromycin | 0.25/0.125 | 4/2 | 0.125 | >32 |
| Oxacillin | 0.5/0.25 | 2 | 0.5/0.25 | 64/16/8 |
| sodium | | | | |
| salt hydrate | | | | |
| Tetracycline | 0.5/0.125 | 0.5/0.125 | 0.25/0.125 | 0.5/0.125/0.06 |
| hydrochloride | | | | |
| Clindamycin | 0.125 | 0.125 | 0.125/0.06/0.03 | >32 |
| hydrochloride | | | | |
| Mupirocin | >32 | >32 | >32 | 0.06/0.03 |
| Cannabidiol | 2/1 | 0.5 | 4/2 | 2/1 |
| (Batch 2) | | | | |

### Example 8

Expanded Panel: Bacteria Minimum Inhibitory Concentration Assays

To assess the potential of Cannabidiol (CBD) for antimicrobial activity against a panel of Gram-positive bacteria.

### Methods

### Compound Preparation

The collaborator supplied sample as dry material. Angela Kavanagh prepared a stock solution at 10 mg/mL in neat DMSO. The highest concentration tested in the assay was 64 μg/mL for bacteria and 128 μg/mL for fungi. 2% DMSO was the final concentration using 1/20 dilution to achieve these concentrations.

### Bacterial Minimum Inhibitory Concentration (MIC) Micro-broth Dilution Assay

The compound was serially diluted in Cation-adjusted Mueller Hinton Broth (CaMHB; BD, Cat. No. 212322) two-fold across the wells of polystyrene (PS) 96-well plates (Corning; Cat. No. 3370), plated in duplicate. All plates had flat bottom wells and were covered with low-evaporation lids.

Bacteria were cultured in CaMHB at 37° C. overnight, then diluted 40-fold and incubated at 37° C. for a further 2-3 h. The resultant mid-log phase cultures were diluted in CaMHB and added to each well of the compound-containing 96-well plates to give a final cell density of 5×10⁵ CFU/mL, and a final compound concentration range of 0.03-64 μg/mL. The plates were covered and incubated at 37° C. for 20 h.

### Bacterial MIC Detection and Analysis

Inhibition of bacterial growth was determined visually, where the MIC was recorded as the lowest compound concentration with no visible growth.

TABLE 25
Tested Compound

| | | | Stock | | Max test | Min test |
|---|---|---|---|---|---|---|
| | Sample | Supplied dry | conc | | conc | conc |
| MCC | name | material (g) | (mg/mL) | Solvent | (μg/mL) | (μg/mL) |
| MCC_009427 | Cannabidiol | 5 | 10 | DMSO | 64 | 0.03 |
| AMRI supply | (CBD) | | | | | |
| Batch ref | | | | | | |
| R0030516 | | | | | | |
| RM342K.0706 | | | | | | |

TABLE 26
Control Compounds

| | | | Stock | | |
|---|---|---|---|---|---|
| | | | cone | | Target |
| | Compound | | (mg/ | | organism |
| MCC | name | MW | mL) | Source | class |
| MCC_000095 | Vancomycin | 1485.71 | 1.28 | Sigma | Gram + |
| | (HCL) | | | 861987 | |
| MCC_000561 | Daptomycin | 1,619.701 | 1.28 | Molekula | Gram + |
| | | | | 64342447 | |
| MCC_000094 | Colistin Sulfate | 1400.63 | 1.28 | Sigma | Gram − |
| | | | | C4461 | |
| MCC_000636 | Polymyxin B | 1301.56 | 1.28 | Sigma | Gram − |
| | Sulfate | | | P0972 | |
| MCC_000191 | Trimethoprim | 290.32 | 1.28 | Sigma | Gram +/− |
| | | | | T7883 | |
| MCC_009395 | Mupirocin | 500.62 | 1.28 | Glentham | Gram + |
| | | | | GA2184 | |
| MCC_008132 | Clindamycin | 504.96 | 1.28 | Glentham | Gram + |
| | hydrochloride | | | GA5034 | |
| | monohydrate | | | | |
| MCC_008383 | Fluconazole | 306.27 | 0.64 | Sigma | Fungi |
| | | | | F8929 | |
| MCC_008384 | 5-fluorocytosine | 129.09 | 0.64 | Sigma | Fungi |
| | | | | F7129 | |

TABLE 27
Tested Organisms

| ID | Species | Strain | Description\| |
|---|---|---|---|
| GP_001:02 | Staphylococcus aureus | ATCC 25923 | MSSA |
| GP_009:01 | Staphylococcus warneri | Clinical isolate | |
| GP_013:01 | Streptococcus pneumoniae | ATCC 33400 | Type strain |
| GP_014:01 | Streptococcus pyopenes | ATCC 12344 | Type strain |
| GP_015:01 | Bacillus cereus | ATCC 11778 | FDA strain |
| | | | PCI 213 |
| GP_016:01 | Bacillus mepaterium | ATCC 13632 | De Bary-KM |
| GP_017:01 | Staphylococcus | ATCC 12228 | FDA strain PCI |
| | epidermidis | | 1200 NRS 231 |
| GP_018:01 | Bacillus subtilis | ATCC 6633 | QC strain |
| GP_020:02 | Staphylococcus aureus | ATCC 43300 | MRSA |
| GP_021:01 | Staphylococcus aureus | ATCC 33591 | MRSA |
| GP_022:01 | Staphylococcus aureus | ATCC 29213 | MSSA |
| GP_023:01 | Streptococcus pneumoniae | ATCC 700677 | MDR |
| GP_024:01 | Enterococcus faecium | ATCC 35667 | Control strain |
| GP_027:01 | Enterococcus faecalis | ATCC 29212 | Control strain |
| GP_033:01 | Staphylococcus epidermidis | NRS 60 | VISA |
| GP_035:01 | Staphylococcus aureus | ATCC 700699, | MRSA, VISA |
| | | NRS 1 | |
| GP_036:01 | Staphylococcus aureus | Clinical isolate | MRSA, |
| | | | DapRSA |
| GP_064:01 | Staphylococcus aureus | NARSA, VRS1 | VRSA |
| GP_197:01 | Staphylococcus epidermidis | ATCC 14990 | Type strain |
| GP_198:01 | Staphylococcus warned | ATCC 27836 | Type strain |
| GP_199:01 | Staphylococcus capitis | ATCC 27840 | Type strain |
| GP_207:01 | Kocuria rosea (formerly | ATCC 31251 | M-1054-1 |
| | Micrococcus roseus | | |
| | Fluppe) | | |

TABLE 28
Summary of Results

| | | Cannabidiol MIC | |
|---|---|---|---|
| | | (Batch 1) | |
| Species | Strain | (μg/mL) | |
| Staphylococcus aureus | ATCC 25923 | 1 | 2 |
| Staphylococcus warneri | Clinical isolate | 2 | 4 |
| Streptococcus pneumoniae | ATCC 33400 | 1 | 2 |
| Streptococcus pyogenes | ATCC 12344 | 1 | 1 |
| Bacillus cereus | ATCC 11778 | 1 | 2 |
| Bacillus megaterium | ATCC 13632 | 1 | 2 |
| Staphylococcus epidermidis | ATCC 12228 | 1 | 2 |
| Bacillus subtilis | ATCC 6633 | 1 | 2 |
| Staphylococcus aureus | ATCC 43300 | 1 | |
| Staphylococcus aureus | ATCC 33591 | 1 | 2 |
| Staphylococcus aureus | ATCC 29213 | 1 | 2 |
| Streptococcus pneumoniae | ATCC 700677 | 1, 2 | 4 |
| Enterococcus faecium | ATCC 35667 | 0.5 | 1 |
| Enterococcus faecalis | ATCC 29212 | 2 | |
| Staphylococcus epidermidis | NRS 60 | 4 | 8 |
| Staphylococcus aureus | ATCC 700699, | 1, 2 | 4 |
| | NRS 1 | | |
| Staphylococcus aureus | Clinical isolate | 2 | 8 |
| Staphylococcus aureus | NARSA, VRS1 | 1 | 2 |
| Staphylococcus epidermidis | ATCC 14990 | 1 | 2 |
| Staphylococcus warneri | ATCC 27836 | 2 | 4 |
| Staphylococcus capitis | ATCC 27840 | 1 | 2 |
| Kocuria rosea | ATCC 31251 | 1 | 2 |

### Results

CBD was active against all Gram-positive strains in a range of 0.5 to 4 μg/mL, except for Staphylococcus epidermidis NDR 60 (GP_033) which was susceptible to CBD at 4 to 8 μg/mL.

Table 28 is a summary of the Minimum Inhibitory Concentration (MIC) range for CBD. The experiment was performed twice in duplicate (n=4) for bacteria. Individual values are shown when they differ between replicates.

### Example 8

Initial efficacy studies focused on screening for antibacterial activity in an ex vivo porcine S. aureus skin infection model. A variety of different CBD compositions ranging from liquids to gels to ointments were evaluated for their ability to kill MRSA at both 1 and 24 h following application (Table 29). Components included differing silicone bases for most preparations (Composition #4-12) with petrolatum (mineral oil jelly i.e. petroleum jelly) tested in Composition #1, transcutol (diethylene glycol monoethyl ether) in Composition #2 and polyethylene glycol (PEG 400/400) in Composition #3. CBD concentrations ranged from 5 to 20% (Table 29).

### Ex-vivo Pig Skin Assay

Porcine tissues, transported on ice, were received 2-5 h after slaughter.

Explant preparation: In RPMI medium containing 2% (v/v) penicillin/streptomycin, a 5 mm biopsy punch was used to cut tissue explants and remaining muscle tissue removed with a sterile scalpel blade. Tissue was antibiotic treated (for decontamination of flora) for 0.5±0.25 h. Explants were rinsed three times with 10±0.5 mL RPMI (no antibiotic, no FBS). Explants were then covered with fresh RPMI (no antibiotic, no FBS) and placed at 4±2° C. for 12±4 h (antibiotic washout). Overnight RPMI was then removed and replaced with 10±0.5 mL fresh RPMI 15±5 min prior to infection.

Bacterial inoculation: Fresh plates were streaked directly from frozen stock within 3 weeks of the experiment. Culture tubes containing Todd Hewitt broth were inoculated with a single colony and placed in a shaking incubator (at 37±2° C., 150±10 rpm) late afternoon the day before the experiment. On the morning of the experiment, 200±50 μL of overnight culture was transferred into 2±0.5 mL fresh Todd Hewitt broth and shaken for 3±1 h at 37° C. Inoculum was then washed to a final concentration of 5×10⁸ CFU mL⁻¹.

Model set up: 6-well plates were set up with 2±0.2 mL RPMI (no antibiotic, no FBS) in each well and a 0.4 μm trans-well insert. Tissue explants were transferred into wells mucosal side up to the insert.

Infection and treatment: Explants were infected with 2±0.5 μl of prepared inoculum (approximately 1×10⁶ CFU/explant or 5×108 CFU mL⁻¹). Explants were incubated at 37±2° C. for 2±0.5 h, then treatments (12 CBD-containing compositions and associated vehicles) administered in triplicate and incubated for at 37±2° C. for 1±0.25 h.

Wash: Post-treatment, 1.0±0.05 mL sterile phosphate-buffered saline (PBS)+2% (w/v) mucin was added to each insert for the appropriate tissue and swirled gently for 5 sec. The liquid suspension was then aspirated, and wells replenished with RPMI (2±0.2 mL RPMI [no antibiotic, no FBS]). Explants were returned to the 37° C. incubator for the indicated post treatment timepoints: 1.0±0.25 h, 24±4 h.

Sample collection: Post-wash (1.0±0.25 h, 24±4 h), tissue was removed from transwells and placed in 500±0.03 μL of neutralizer (30 mg/mL bovine serum albumin). Samples were sonicated and vortexed (30±5 sec vortex, 120±6 sec sonicate, 30±5 sec vortex). Samples were then plated neat or diluted in sterile PBS. 50±2 μL of sample was plated with a spiral plater on mannitol salt agar, and plates incubated for 24-48 h at 37±2° C. The following day, colonies were counted with an automated plate counter and CFU counts transformed to Log₁₀(CFU/explant).

TABLE 29
Topical Compositions

| | | | | | | | | | |
|---|---|---|---|---|---|---|---|---|---|
| Composition Number | 1 | 2 | 3 | 4 | 5 | 6 | 7 | 10 | 12 |

Ingredients (% w/w)

| | | | | | | | | | |
|---|---|---|---|---|---|---|---|---|---|
| Dow Q7-9180 Silicone Fluid 0.65 cst | 0 | 0 | 0 | 92 | 44.5 | 13.32 | 3.5 | 47 | 33.5 |
| Dow Q7-9120 Silicone Fluid | 0 | 0 | 0 | 1 | 0 | 2.02 | 2.5 | 0 | 0 |
| 12500 cst | | | | | | | | | |
| Dow 9045 Silicone Elastomer Blend | 0 | 0 | 0 | 0 | 25 | 69.68 | 79.04 | 0 | 0 |
| Dow Corning BY 11-030 | 0 | 0 | 0 | 0 | 0 | 0 | 0 | 0 | 15 |
| Arlamol PS15E | 0 | 14.1 | 0 | 2 | 8 | 4.98 | 5.27 | 10 | 0 |
| Dow Corning 9041 Elastomer Blend | 0 | 0 | 0 | 0 | 0 | 0 | 0 | 0 | 0 |
| Compritol 888 ATO | 0 | 0 | 0 | 0 | 10 | 0 | 0 | 0 | 0 |
| Petrolatum | 80 | 0 | 0 | 0 | 2.5 | 0 | 0 | 0 | 0 |
| Castorwax | 0 | 0 | 0 | 0 | 0 | 0 | 0 | 0 | 0 |
| Isopropyl Alcohol | 0 | 3.4 | 0 | 0 | 0 | 0 | 0 | 3 | 0 |
| Isopropyl Myristate | 0 | 0 | 0 | 0 | 0 | 0 | 0 | 0 | 0 |
| Plural diisostearique | 0 | 0 | 0 | 0 | 0 | 0 | 0 | 0 | 0 |
| Monosteol (PG Stearate) | 0 | 0 | 0 | 0 | 0 | 0 | 0 | 0 | 0 |
| Transcutol | 0 | 62.5 | 0 | 0 | 0 | 0 | 0 | 20 | 30 |
| PEG 400 | 0 | 0 | 50 | 0 | 0 | 0 | 0 | 0 | 0 |
| PEG 4000 | 0 | 0 | 30 | 0 | 0 | 0 | 0 | 0 | 0 |
| Water | 0 | 0 | 0 | 0 | 0 | 0 | 0 | 0 | 1.5 |
| Cannabidiol | 20 | 20 | 20 | 5 | 10 | 10 | 10 | 20 | 20 |

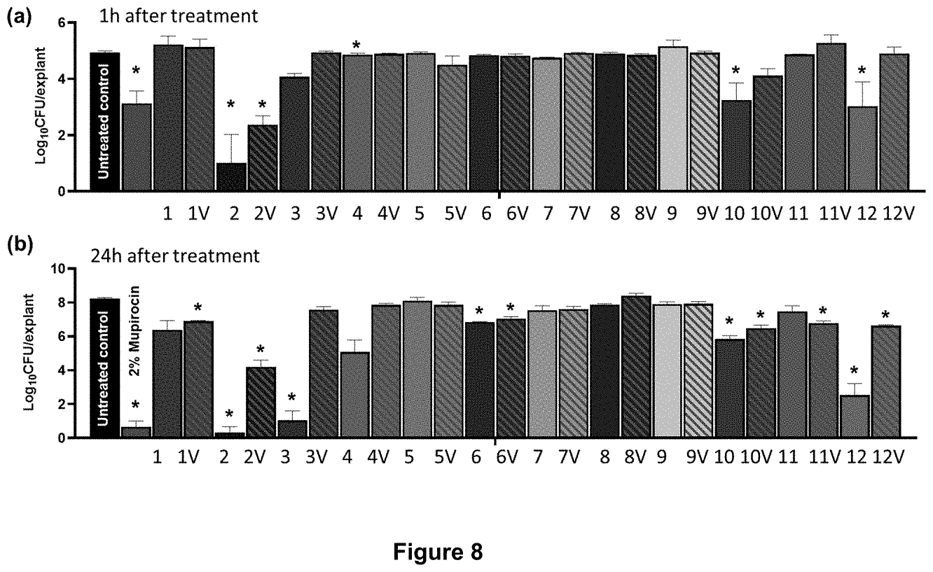

Efficacy was very composition-dependent, and some composition vehicles had modest to good antimicrobial activity on their own (e.g. composition 2, with a high content of transcutol and 3.4% isopropyl alcohol). Results are provided in FIG. 8.

The lack of activity in some compositions was due to the CBD not being sufficiently released from the composition.

Good activity (2- to 3-log reduction in colony-forming units [CFU] after 1 h, >5 log reduction at 24 h) was consistently observed with Compositions #3 and #12, but not their corresponding vehicles. Composition #3 is a PEG-based composition, which matches that used for Bactroban™ (mupirocin) ointment, containing 20% CBD. Composition #12 has a mixture of a silicone fluid (polydimethylsiloxane liquid) and transcutol combined with a gelling agent (Dow Corning BY 11-030) and a small amount of water, again with 20% CBD.

TABLE 30
Composition of the BTX 1801 Active and
Vehicle Control Compositions (w/w)
BTX1801 Composition

| | BTX | BTX | BTX | BTX 1801 |
|---|---|---|---|---|
| | 1801 | 1801 | 1801 Gel | Ointment |
| Ingredients (% w/w) | Gel | Ointment | Vehicle | Vehicle |
| Hexamethyldisiloxane | 33.5 | 0 | 41.9 | 0 |
| Dow BY 11-030 | 15 | 0 | 18.8 | 0 |
| Transcutol | 30 | 0 | 37.5 | 0 |
| Polyethylene glycol | 0 | 80 | 0 | 100 |
| 400/4000 (mixture) | | | | |
| Water | 1.5 | 0 | 1.9 | 0 |
| Cannabidiol (CBD) | 20.0 | 20.0 | 0 | 0 |
| Total | 100.0 | 100.0 | 100.0 | 100.0 |

### Example 9

### General Microbial Assay Protocol

Test compositions were provided by Botanix/Formulytica and designated with a number of the form F###-#-##/L###-#-##, where the “F” number refers to a particular composition and the “L” number refers to a manufacturing batch. All experiments dated prior to Dec. 5, 2019 were performed using compositions with an “L” number of the form L144-2-##; all experiments dated after Dec. 5, 2019 were performed using compositions with an “L” number of the form L144-3-##.

Each experiment had two timepoints: 1±0.25 hours, 24±2 hours with 3 explants for each strain/treatment/timepoint combination.

Bacterial species and strain: Staphylococcus aureus MRSA ATCC 43300, high-level mupirocin-resistant MRSA strains 329 and 993, low-level mupirocin-resistant MRSA strain 815. Mupirocin-resistant strains characterized in a prior manuscript: Antimicrob. Agents Chemother. 59, 2765-2773 (2015).

The tissue type used was Porcine skin tissue (PST).

Neutralizer: 500 μL or 1 mL 30 mg/mL BSA for all CBD-containing vehicles; 500 pg amberlite beads (XAD-40) in 1 mL PBS was used to neutralize mupirocin.

### Skin and Explant Preparation

Porcine skin tissue (PST) from a pig harvested for meat 2-5 hours prior to arrival in lab was transported to the laboratory on ice. A section or sections of skin approximately 8 cm×8 cm was cleaned to remove gross contamination and shaved. 5 mm biopsy punches were used to cut tissue explants and remaining muscle tissue was removed with a sterile scalpel blade.

Explants were soaked in RPMI+2% (v/v) penicillin/streptomycin for 24-48 hours at 4±2° C. to reduce presence of normal flora. Explants were rinsed twice with fresh RPMI (no antibiotics) and soaked for 14±3 hours at 4±2° C. to remove antibiotics. Immediately prior to use in assay explants were washed once more and soaked in RPMI (no antibiotics) for 30±10 minutes at 37±2° C.

Explants were placed into 6-well cell culture plates atop 0.4 μm trans-well inserts with 2±0.5 mL RPMI below the insert.

### Bacteria Preparation

A plate was streaked for isolation directly from frozen stock onto a blood agar plate (BAP) within three weeks of experiment date. A culture tube containing Todd Hewitt Broth (THB) containing a sub-lethal amount of mupirocin was inoculated with a single colony from the BAP and placed in shaking incubator (37±2° C., 200±10 rpm) in the late afternoon the day before the experiment. This broth was prepared by diluting 2% mupirocin ointment 1:100 in THB.

The day of the experiment, 200±50 μL of the overnight culture was transferred into 2±0.5 mL fresh THB, and shaken for 3±1 hour at 37° C. An inoculum of approximately 5×108 CFU/mL in RPMI with washes by centrifugation at ˜20,000×g followed by removal of supernatant and resuspension of pellet. Inoculum was generated by diluting the passaged culture to a concentration of ˜5×10⁸ CFU/mL in RPMI medium. This was generally a 1:4 dilution, corresponding to an optical density at 600 nm of ˜0.6. This wash step was completed in full twice, with the third resuspension used as inoculum.

Final inoculum was measured quantitatively by preparing a 1:10,000 dilution (2×1:100 serial dilution) and plating 50 μL of this dilution on MSA.

### Infection

Pipet 2 μL of ˜5×108 CFU/mL inoculum onto each explant (˜1×106 CFU/VVound Bed). Incubate at 37±2° C. for 2±0.25 h.

### Treatment and Wash

Explants were treated with 100 μL of appropriate composition with no composition applied to the Growth Control (GC) explants. Explants were incubated at 37±2° C. for 1±0.25 h.

Explants were washed using 1.0±0.05 mL sterile PBS+2% (w/v) mucin into each insert. Mucin was introduced directly onto each explant in the well. Swirl gently for 5±2 seconds. Mucin and residual treatment was aspirated and mechanically removed as necessary.

Media below trans-wells (RPMI without antibiotics) was removed and replaced with a fresh 2±0.5 mL. Explants were returned to 37±2° C. for 1±0.25 h or 24±2 h.

### Sample Collection

At the appropriate time, explants were removed and placed into neutralizer (as described above).

Explants were treated with a vortex/sonicate/vortex series to liberate bacteria (30±5 sec vortex, 120±6 sec sonicate, 30±5 sec vortex). 50±2 μL of each sample was plated on mannitol salt agar plates using a spiral plater (neat, at a 1:100 dilution, or at a 1:10,000 dilution). Plates incubated for 24-48 h at 37° C., enumerated using an automated plate counter, and transformed into Log10(CFU/explant).

### Data and Statistical Analysis

Plate counts were imported into Prism (Graphpad), and data was graphed as mean with standard error of the mean (SEM). In general, weekly data was analysed using Prism by separating the two timepoints using the “multiple comparisons” of a one-way ANOVA analysis with Holm-Sidak post- correction.

Combined data sets were generated by storing all data in a Microsoft Excel workbook. Overall means were calculated by multiplying each experimental mean by the number of samples in that experiment (weighted mean), summing this value for each experiment, and dividing by the total number of samples.

The standard deviation for the combined data set was calculated by an application of the law of total variance. Variance for each experimental data set was calculated by squaring the standard deviation (as calculated by Excel) added to the square of the difference between the mean for that experiment and the total weighted mean; this combined value was multiplied by the number of samples for each experiment. The standard deviation of the full data set was calculated by taking the square root of the sum of these variances divided by the total number of samples.

Standard error of the mean was calculated by dividing the standard deviation by the square root of the total number of samples. Statistical significance was determined by importing the data into Prism (Graphpad) and using the “multiple comparisons” of a 2-way ANOVA analysis with a Dunnett's post-correction.

### Neutralization Protocol

### Neutralization Protocol Background

Explant and Bacteria preparation identical to that described in “General Antimicrobial Assay Protocol” above. Final inoculum was prepared as a suspension of 5×104 CFU/mL in PBS.

### Challenge Preparation

Eppendorf tubes (1.7 mL) containing 1 mL of PBS for the negative control, 500 μg Amberlite Beads (XAD-40) in 1 mL PBS (for mupirocin), or 1 mL of 30 mg/mL BSA in PBS pH=7.4 for all CBD-containing compositions and their vehicles were spiked with ˜1×103 (3 log) CFU of the S. aureus ATCC 43300 bacteria primarily used in the project (20 μL of the 5×104 CFU/mL inoculum).

Explants were prepared as described in “Skin and Explant Preparation” above and treated as described in the “Treatment and Wash” and “Sample Collection” above.

### Sample Collection and Quantitation

Explants were placed in the prepared 1.7 mL Eppendorf tubes containing ˜1×103 and subjected to the same neutralization and plating protocol outlined in the “Sample Collection” above. Plates were counted after ˜36 hours. Any treatment group with a log reduction from growth control of less than or equal to 0.2 was considered to have passed (per ASTM E1054).

### Minimum Inhibitory Concentration (MIC) Microtiter Broth Dilution Protocol

### MIC Bacterial Preparation

Strains used: ATCC 29213 (standard control), ATCC 43300, low-level mupirocin-resistant isolate 815, high-level mupirocin-resistant isolage 329, high-level mupirocin-resistant isolate 993.

All strains prepared as described in “Bacterial Preparation” section above with the following differences: inocula were prepared in Müller-Hinton broth (MHB) rather than RPMI, and were diluted 1:10,000 following wash for an inoculum concentration of ˜5×104 CFU/mL.

### Plate Preparation

Using a multichannel pipet, 100 μL of MHB was added to each of the wells of the 96-well plates (round bottom/U-bottom). Stock solutions of of antibiotic were prepared at 2× the highest assay concentration (mupirocin at 4096 μg/mL and CBD at 100 μg/mL). 100 μL of this broth was added to each of the broth-containing wells in column 1 of the plate, and mixed by pipetting the full volume 6-7 times, for a final concentration of 2048 μg/mL mupirocin or 50 μg/mL CBD. 100 μL of the broth from column 1 was added to column 2 and mixed by pipetting 100 μL 6-7 times. This was repeated through column 11 (2 μg/mL mupirocin or 0.049 μg/mL CBD). Column 12 was left without antimicrobial.

### Experiment Setup

5 μL of each inoculum was added to the appropriate wells (various wells were left without inoculation and used to correct the absorbance of the CBD-containing wells and as negative controls). Plate was incubated at 37° C. for 18-24 hours. Plate was read by a plate reader at 600 nm.

### Data Analysis

Inhibition was determined to have occurred in the first well with a difference in OD600 of <0.05 from the uninfected wells with the same concentration of antimicrobial, provided inhibition growth continued through all higher concentrations. The experiment was performed twice for each antimicrobial, and data reported as a range of the two values obtained.

### Irritation (MTT) Protocol

### MTT Tissue Preparation

Explants were prepared as described in “Skin and Explant Preparation” above and treated as described in the “Treatment and Wash” and “Sample Collection” above. Explants were placed in 6-well plates on sterile gauze soaked in RPMI+2% (v/v) penicillin-streptomycin rather than trans-wells.

### Treatment and Experimental Design

10 μL of composition or control was added to the top of explants and incubated for 24±2 hours. A “Treatment” was one of the CBD-containing compositions or the associated vehicle. The controls were PBS (pH=7.4) as a non-irritating control, 10% Tween-20 in distilled water as a non-irritating detergent, 1% Triton in distilled water as a mildly irritating detergent, and 5% SDS in distilled water as a highly irritating detergent. All controls were used in all assays.

### MTT Assay

Following incubation with treatment, explants were added to 100 μL MTT reagent and incubated for 1.5±0.5 h at 37±2° C. Following incubation with MTT reagent, explants were added to 100 μL of de-stain (0.1 M HCl in 2-propanol) and incubated for 20±4 hours at 4±2 ° C.

### Data Collection and Analysis

Explants were removed from wells, and remaining de-stain was read by absorbance at 570 nm and 600 nm. The 570 nm absorbance corresponds to the MTT signal and the 600 nm signal shows any nonspecific blockage of light (such as by leftover tissue). Data was normalized to non-irritating controls (PBS and/or 10% Tween-20).

### Results

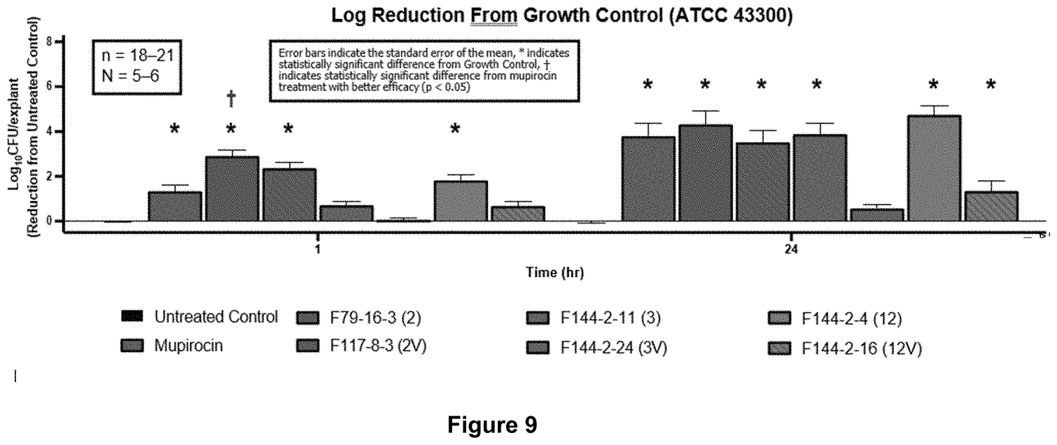

Of the original 12, the best compositions were determined to be F79-16-3 (liquid), F144-2-11 (ointment), and F144-2-4 (gel). At 1 hour, F79-16-3 (2.9 log reduction) and F144-2-4 (1.8 log reduction)were most effective against S. aureus ATCC43300 (FIG. 8, FIG. 9).

At 24 hours, all three compositions were highly effective, with 4.3, 3.9, and 4.7 log reductions, respectively. The vehicle for F79-16-3 was comparable in efficacy toward ATCC 43300 (FIG. 8, FIG. 9).

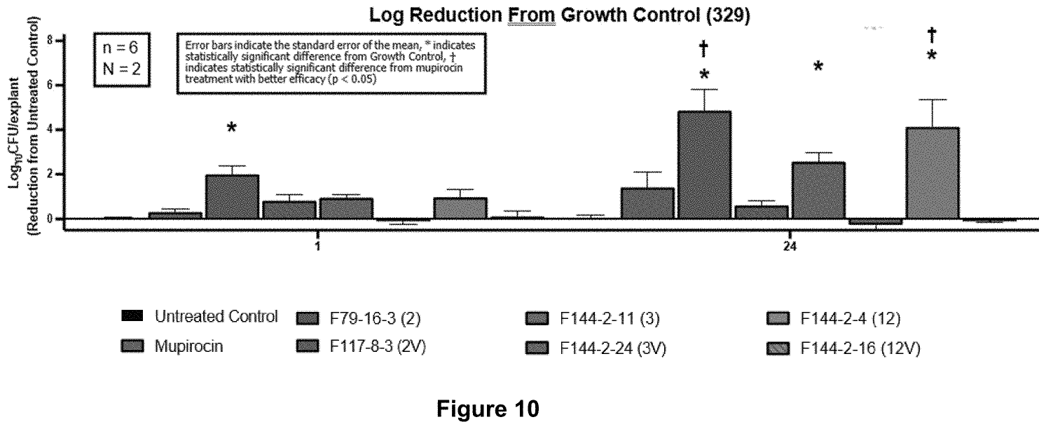

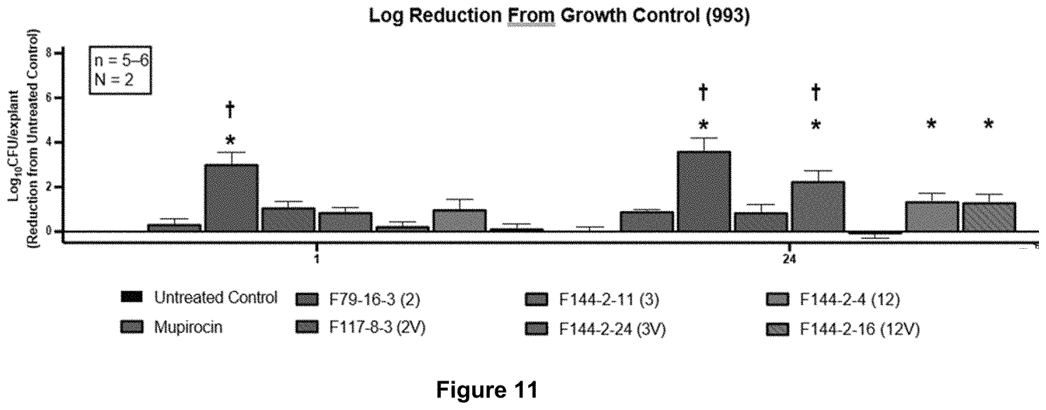

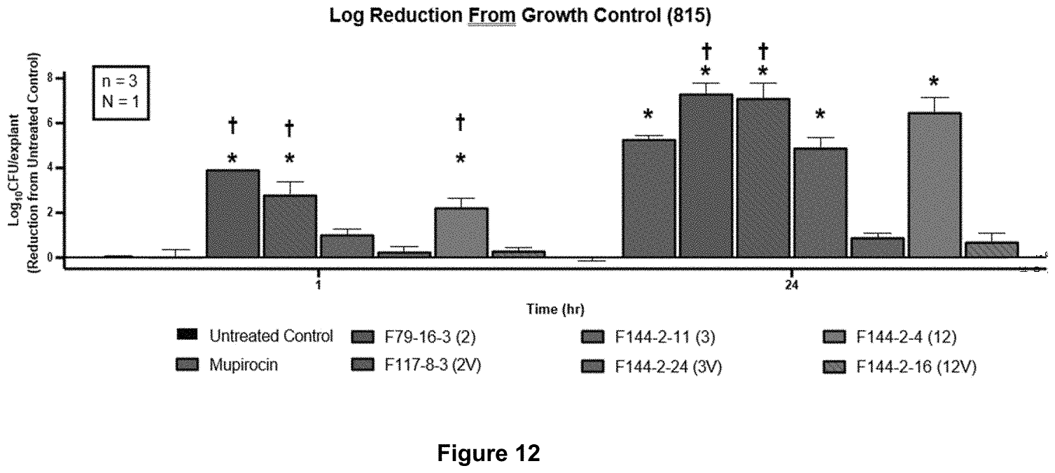

All compositions were comparably effective against mupirocin-resistant MRSA strains (FIG. 10-12).

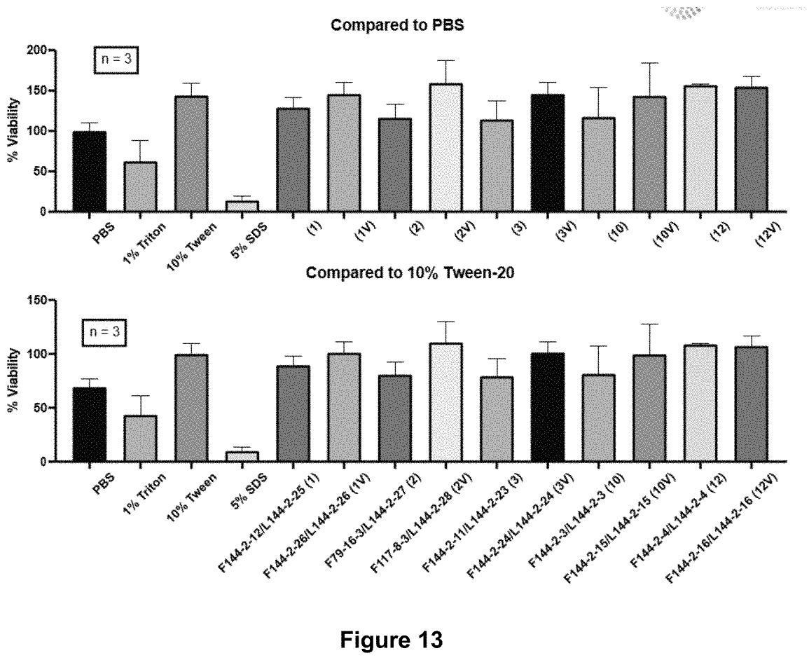

All CDC-containing compositions were non-irritating to ex vivo porcine skin (FIG. 13).

Of the 12 analysed, the best compositions, as measured by the antimicrobial assay, are F79- 16-3 (liquid), F144-2-11 (ointment), and F144-2-4 (gel). All three compositions were more effective than mupirocin against highly mupirocin-resistant (MIC 256 to 2048 ug/mL) MRSA strains (329 and 993). Compositions F79-16-3 and F144-2-4 appear more effective than mupirocin against low-level resistant strain numbered 815.

### Example 10

### General Microbial Assay Protocol

Test compositions were provided by Botanix/Formulytica and designated with a number of the form F###-#-##/L###-#-##, where the “F” number refers to a particular composition and the “L” number refers to a manufacturing batch.

Each experiment had two timepoints: 1±0.25 h, 24±2 h with 3 explants for each strain/treatment/timepoint combination. The bacterial species and strain: Staphylococcus aureus ATCC 43300.

### Skin and Explant Preparation

Porcine skin tissue (PST) from a pig harvested for meat 2-5 h prior to arrival in lab was transported to the laboratory on ice. A section or sections of skin approximately 8 cm×8 cm was cleaned to remove gross contamination and shaved. 5 mm biopsy punches were used to cut tissue explants and remaining muscle tissue was removed with a sterile scalpel blade.

Explants were rinsed twice with 15±5 mL of RPMI+2% (v/v) penicillin/streptomycin+0.5 mg/L amphotericin B (RPMI+ABXF). Explants were soaked in fresh 15±5 mL RPMI+ABXF for 1±0.25 h at 37±2° C. to reduce presence of normal flora. Explants were rinsed twice with fresh RPMI (no antibiotics). Explants were soaked in fresh RPMI for 1±0.25 h at 37±2° C. to remove antibiotics.

Immediately prior to use in assay explants were washed twice more with 15±5 mL of RPMI. Explants were placed into 6-well cell culture plates atop 0.4 μm trans-well inserts with 2±0.5 mL RPMI below the insert.

### Bacteria Preparation

A plate was streaked for isolation directly from frozen stock onto a blood agar plate (BAP) or mannitol salt agar (MSA) plate within three weeks of experiment. A culture tube containing Todd Hewitt Broth (THB) was inoculated with a single colony from the BAP and placed in shaking incubator (37±2° C., 200±10 rpm) in the late afternoon the day before the experiment.

The day of the experiment, 200±50 μL of the overnight culture was transferred into 2±0.5 mL fresh THB, and shaken for 3±1 h at 37° C. An inoculum of approximately 5×108 CFU/mL in RPMI with washes by centrifugation at 20,000×g followed by removal of supernatant and resuspension of pellet. Inoculum was generated by diluting the passaged culture to a concentration of ˜5×108 CFU/mL in RPMI medium. This was generally a 1:4 dilution, corresponding to an optical density at 600 nm of ˜0.6. This wash step was completed in full twice, with the third resuspension used as inoculum.

Final inoculum was measured quantitatively by preparing a 1:10,000 dilution (2×1:100 serial dilution), plating 50 μL of this dilution on MSA, and enumerating colonies.

### Infection

Inoculum (2±0.5 μL of ˜5×108 CFU/mL S. aureus) was pipetted onto each explant (˜1×106 CFU/explant). Incubated at 37±2° C. for 2±0.25 h.

### Treatment

Explants were treated with 100 pL of appropriate composition with no composition applied to the Growth Control (GC) explants. Explants were incubated at 37±2° C. for 1±0.25 h.

Explants were washed using 1.0±0.05 mL sterile PBS+2% (w/v) mucin into each well. Mucin was introduced directly onto each explant in the well and swirled gently for 5±2 seconds. Mucin and residual treatment was aspirated and mechanically removed as necessary.

Media below trans-wells (RPMI without antibiotics) was removed and replaced with a fresh 2±0.5 mL. Explants were returned to 37±2° C. for 1±0.25 h or 24±2 h.

### Sample Collection

At the appropriate time explants were removed and placed into neutralizer (as described above). Explants were treated with a vortex/sonicate/vortex series to liberate bacteria (30±5 sec vortex, 120±6 sec sonicate, 30±5 sec vortex).

50±2 μL of each sample was plated on mannitol salt agar plates using a spiral plater (neat, at a 1:100 dilution, or at a 1:10,000 dilution). Plates incubated for 24-48 h at 37° C., enumerated using an automated plate counter, and transformed into Log10(CFU/explant).

### Data and Statistical Analysis

Plate counts were imported into Prism (Graphpad), and data was graphed as mean with standard error of the mean (SEM). In general, weekly data was analyzed using Prism by separating the two timepoints using the “multiple comparisons” of a one-way ANOVA analysis with Holm-Sidak post-correction.

Combined data sets were generated by storing all data in a Microsoft Excel workbook. Overall means were calculated by multiplying each experimental mean by the number of samples in that experiment (weighted mean), summing this value for each experiment, and dividing by the total number of samples.

The standard deviation for the combined data set was calculated by an application of the law of total variance. Variance for each experimental data set was calculated by squaring the standard deviation (as calculated by Excel) added to the square of the difference between the mean for that experiment and the total weighted mean; this combined value was multiplied by the number of samples for each experiment. The standard deviation of the full data set was calculated by taking the square root of the sum of these variances divided by the total number of samples. Standard error of the mean was calculated by dividing the standard deviation by the square root of the total number of samples. Statistical significance was determined by importing the data into Prism (Graphpad) and using the “multiple comparisons” of a 2-way ANOVA analysis with Dunnett's post-correction.

TABLE 31
Treatments

| Name | Identifying number |
|---|---|
| 5% Ointment | F/L 144-5-1 |
| 5% Ointment (Vehicle) | F/L 144-5-2 |
| 10% Ointment | F/L 144-5-3 |
| 10% Ointment (Vehicle) | F/L 144-5-4 |
| 15% Ointment | F/L 144-5-5 |
| 15% Ointment (Vehicle) | F/L 144-5-6 |
| 3 (20% Ointment) | F144-2-11 (B) 144-3-05 |
| 3 V (20% Ointment Vehicle) | F144-2-24 (B) 144-3-06 |
| 5% Gel | F/L 144-5-7 |
| 5% Gel (Vehicle) | F/L 144-5-8 |
| 10% Gel | F/L 144-5-9 |
| 10% Gel (Vehicle) | F/L 144-5-10 |
| 15% Gel | F/L 144-5-11 |
| 15% Gel (Vehicle) | F/L 144-5-12 |
| 12 (20% Gel) | F144-2-04 (B) 144-3-07 |
| 12 C (20% Gel Vehicle) | F144-2-16 (B) 144-3-08 |

### Results

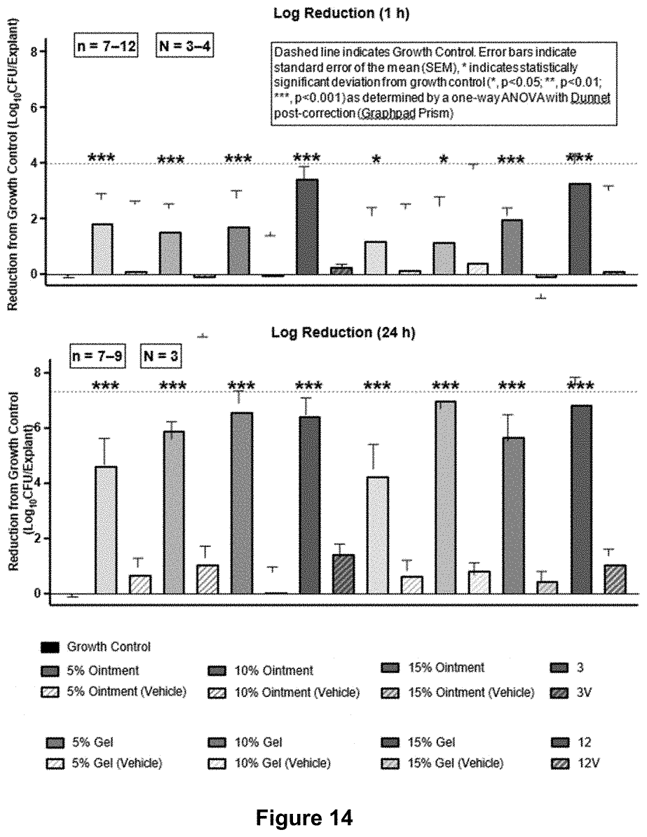

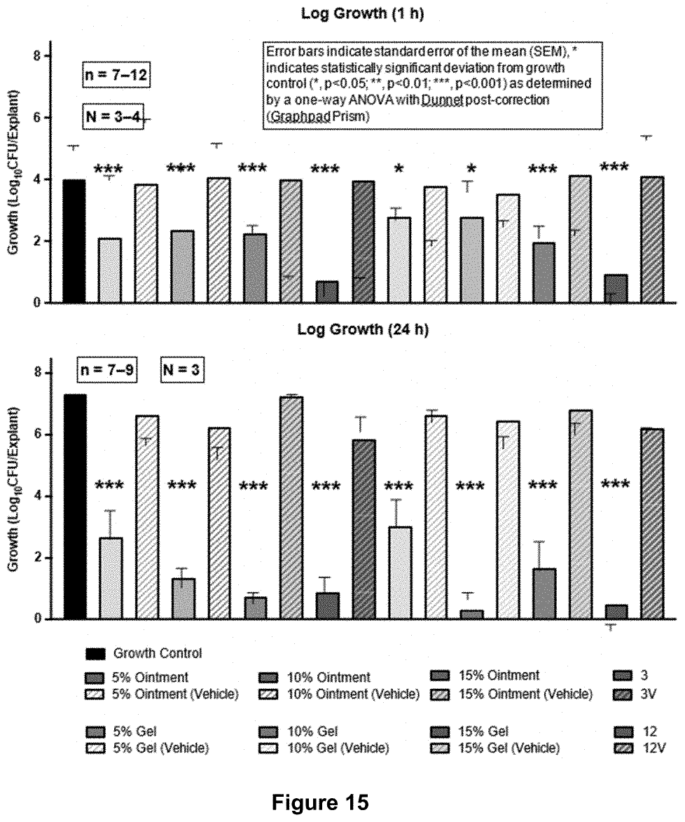

All CBD-containing treatments resulted in statistically significant (p<0.05) reduction from growth control at both 1 h and 24 h. No vehicle resulted in a statistically significant reduction from growth control at neither 1 h nor 24 h. The largest aggregate reduction from a vehicle treatment was the 20% ointment vehicle, which resulted in ˜1.4 Log reduction (FIG. 14, FIG. 15).

The 20% CBD composition was clearly the most effective concentration at 1 h (˜3.4 Log reduction), other treatments did not form a curve at (˜1.8, ˜1.5, and ˜1.7 Log reduction for 5%, 10%, and 15% compositions respectively); 20% CBD ointment was significantly different (p<0.05) from the 5%, 10%, and 15% compositions. At 24 h, the composition effectiveness approximately correlated with CBD-percentage (in ascending order 5% to 20%: ˜4.6, ˜5.9, ˜6.5, ˜6.4 Log reduction); no non-vehicle treatments were significantly different from another.

At 1 h, the effectiveness of the gel compositions approximately correlated with CBD-percentage (in ascending order: ˜1.2, ˜1.2, ˜2, ˜3.3 Log reduction), and the difference between the 5% and 20% is statistically significant; 20% CBD gel was significantly different (p<0.05) from the 5%, 10%, and 15% compositions. At 24 h, there was more variation (in ascending order: ˜4.3, ˜7.0, ˜5.7, ˜6.8), though there appears to be increased efficacy based on concentration; the difference between the 5% and 10% gel compositions was statistically significant (p=0.0335).

The data indicate general trends that higher CBD-concentration results in more antimicrobial effectiveness for both compositions at the 1 h and 24 h post-treatment timepoints.

### Example 11

A Randomised, Double-Blind, Vehicle-Controlled Study to Evaluate Safety, Tolerability, and Efficacy of Two Dosage Forms of BTX 1801 Applied Twice Daily for Five Days to the Anterior Nares of Healthy Adults Nasally Colonised with Staphylococcus aureus

This randomised, double-blind, vehicle-controlled study will evaluate the safety, tolerability and efficacy of two dosage forms of BTX 1801 compared to their corresponding vehicles, applied BID for 5 days to the anterior nares of healthy adults nasally colonised with SA. Approximately 60 participants will be randomised 2:2:1:1 (20 participants to BTX 1801 20% (w/w) Ointment, 20 participants to BTX 1801 20% (w/w) Gel, 10 participants to BTX 1801 Vehicle Ointment, and 10 participants to BTX 1801 Vehicle Gel).

Participants will attend 2 screening visits to determine SA nasal colonisation via culture of anterior nares swabs at Screening Visit 1 (Days −28 to −14) and Screening Visit 2 (Days −11 to −4). Participants will be identified as persistent or intermittent carriers following results of Baseline nasal cultures (Visit 3; Day 1).

TABLE 32
Identification of Intermittent versus Persistent Colonisation Status

| Anterior nares culture for S. aureus | | | Nasal Colonisation |
|---|---|---|---|
| Screening Visit 1 | Screening Visit 2 | Baseline Visit 3 | Status |
| Negative | — | — | Not Colonised¹ |
| Positive | Negative | — | Not Colonised² |
| Positive | Positive | Negative | Intermittent³ |
| Positive | Positive | Positive | Persistent³ |

¹Not eligible for Screening Visit 2
²Not eligible for Baseline Visit 3
³Intermittent and Persistent carriers are eligible for the randomisation

Eligible participants will receive their first application of study drug at the site on Day 1 and will self-apply their other dose at home. Participants will be treated for 5 consecutive days (Visits 3-7), and return for follow-up visits on Days 8,12 and 28 (Visits 8-10).

Throughout the study, safety will be monitored by TEAEs, local tolerability (TNSS and macroscopic nasal examination), clinical laboratory assessments, physical examination, and vital signs. Concomitant medications will be recorded throughout the study.

Blood samples to determine CBD plasma concentrations will be collected pre-dose (on Days 1 (Baseline), and on Days 2 and 5 (during treatment). Details of the participant's study drug application (including the date, time, and amount) will be recorded in study drug application diary. Anterior nares swabs to measure SA nasal colonisation will also be collected at all follow-up visits (Days 8, 12 and 28; Visits 8-10).

### Primary Endpoints

To assess the safety and tolerability of BTX 1801 relative to Baseline for the following parameters:
Treatment-emergent adverse events (TEAEs)
Total Nasal Symptom Score (TNSS)
Macroscopic nasal examination
Clinical laboratory assessments and to assess the percentage of persistent SA carriers with a negative nasal culture for SA on Day 12

### Secondary Endpoints

To evaluate changes in nasal SA colonisation associated with study drug application as follows:
of persistent SA carriers with a negative nasal culture for SA on Days 8 and 28
of participants with a negative nasal culture for MRSA on study Days 8, 12 and 28
of participants who have nasal recolonisation with SA on study day 12 and/or 28 after a negative nasal culture on Day 8 and to assess the plasma levels of study drug taken pre-dose at Baseline and Days 2 and 5.
   Inclusion criteria

To be included in the study, participants must meet the following inclusion criteria.
Participant is of either gender of ≥18-65 years of age.
Participant is in good general health without clinically significant respiratory, gastrointestinal, renal, hepatic, haematological, lymphatic, neurological, cardiovascular, psychiatric, musculoskeletal, genitourinary, immunological, dermatological, malignant disease, or connective tissue diseases or disorders, as determined by the investigator.
Confirmed to be nasal SA carriers, defined as having 2 separate SA positive cultures from anterior nares swabs during screening.

### Exclusion Criteria

If a participant meets any of the following exclusion criteria, they may not participate in the study.

Methicillin-susceptible and methicillin-resistant Staphylococcus aureus decolonisation attempt in the 6 months prior to screening.

Nasal surgery within 3 months prior to Screening Visit 1.

Evidence of active rhinitis, sinusitis or upper respiratory tract infection at Screening Visits 1 or 2 or Baseline Visit.

Participant has any significant active infection.

Participant has used topical or systemic antibiotics within 4 weeks of Baseline.

Negative nasal culture for SA at Screening Visit 1 or 2.19. Planned use of any nasal applied medication (other than study drug) during the study.

### Participant Enrolment

Participants will be randomised 2:2:1:1 to receive BTX 1801 20% (w/w) Ointment, BTX 1801 20% (w/w) Gel, BTX 1801 Vehicle Ointment, to BTX 1801 Vehicle Gel).

### Study Drug

Study drug will be provided to the study site by Formulytica Pty Ltd in Mulgrave, Victoria, Australia. Initial shipments will be made to supply the study site prior to enrolment of the first participant. Additional supplies will be made available as needed based on participant enrolment.

### BTX 1801 Compositions

Botanix Pharmaceuticals' BTX 1801 contains the active pharmaceutical ingredient, CBD.

Two compositions of BTX 1801 and their corresponding Vehicle-control compositions will be provided to the study site in 20 g aluminium laminate tube with a 15 g fill. The excipients include hexamethyldisiloxane, hexamethyldisiloxane (Dow 9180), Transcutol P (diethylene glycol monoethyl ether), cyclopentasiloxane+polyethylene glycol (PEG)/polypropylene glycol (PPG)-10/19 dimethicone blend (Dow BY 11-030), PEG 400, PEG 4000 and water which have been used extensively in other topical products. The active BTX 1801 study products are a clear to light pink solution with a 20% (w/w) concentration of CBD. The compositions of the BTX 1801 compositions and their corresponding Vehicle-controls are presented in the table below.

TABLE 31
Composition of the BTX 1801 Active
& Vehicle Control Compositions

| | BTX 1801 Composition | | | |
|---|---|---|---|---|
| | BTX 1801 | BTX 1801 | BTX 1801 | BTX 1801 |
| Ingredients (% w/w) | Gel | Ointment | Vehicle Gel | Vehicle |
| Hexamethyldisiloxane | 33.5 | 0 | 41.9 | 0 |
| Dow BY 11-030 | 15 | 0 | 18.8 | 0 |
| Transcutol | 30 | 0 | 37.5 | 0 |
| Polyethylene glycol | 0 | 80 | 0 | 100 |
| 400/4000 (mixture) | | | | |
| Water | 1.5 | | 1.9 | 0 |
| Cannabidiol (CBD) | 20.0 | 20.0 | 0 | 0 |
| Total | 1000 | 1000 | 1000 | 1000 |

Each gram of the BTX 1801 may contain up to 200 mg of CBD. The maximum daily exposure following application of 0.25 g to each nostril BID of each BTX 1801 composition is ˜200 mg of CBD.

Study drug will be applied by a study staff member different from the evaluator so that clinical assessments are blinded.

### Dosing and Administration

Two 20 g tubes of study drug will be assigned to each participant. One tube will be dispensed to each participant on Day 1 and will be sufficient supply for the 5 day application stage. The second tube will remain at the study site as back-up, if needed. Study drug is applied BID, one dose will be applied under the supervision of unblinded study site staff each day (Days 1-5) and the participants will self-apply the other dose of study drug at home. Participants will be instructed to bring their study drug to the study site each day.

Participants will be instructed on how to apply study drug when not at the clinical site. Each application of study drug will occur approximately at the same time in the morning with the second application approximately 12 hours later.

The dose for all participants will be 0.25 g of study drug applied BID to each anterior nare (0.5 g per nare per day). Participants will dispense a finger-top-unit (FTU) of study drug by squeezing a line of study drug from the tip of their index finger to the first crease and instructed to apply to one of the anterior nares by gently rolling the finger-tip over inner surface of the nare. Following application to each nare, participants will be instructed to gently pinch the nose intermittently for approx. 1 minute to ensure distribution of study drug within the anterior nares.

No escalation of dose will occur. Participants will receive BID application of study drug for a total of 10 doses.

### Screening Timeline

### Visit 1: Days -28 to -14 (Screening)

At the first Screening Visit (Visit 1), participants will review and sign a pre-screening ICF for the collection of anterior nares swabs to test for SA colonisation and attesting to their knowledge that if the nasal swab is negative for SA, he/she will not be invited to proceed to Screening Visit 2. The following study specific procedures will occur at the Screening Visit 1:
Pre-screening informed consent
Anterior nares swabs for SA culture

Confirmation of SA positive culture from anterior nares swabs is required before determining whether participant is eligible for Screening Visit 2.

### Visit 2: Days -11 to -4 (Screening)

At the second Screening Visit (Visit 2), the following procedures/assessments will be conducted:
Informed consent for main study
Inclusion/exclusion criteria review
Demographic information collection
Medical & medication history collection
Physical examination, including body measurements (body weight and height)
Anterior nares swabs for SA culture

Confirmation of SA positive culture from anterior nares swabs collected at this visit is required before determining whether the participant is eligible for the Baseline Visit.

### Visit 3: Day 1 (Baseline; Start of Treatment)

On Day 1 (start of study drug use), the following procedures/assessments will be conducted prior to study drug intake:
Randomisation to study drug group
   Following Randomisation the Following will be Performed
TNSS
Macroscopic nasal examination
Anterior nares swabs for SA culture (swabs to be retained)
Blood collection for study drug level prior to study drug application
Weigh and dispense first tube of study drug. One tube of study drug to be dispensed to each participant; the same tube will be taken home and returned to the study site each day for compliance monitoring and dosing at the site
Train participant in proper application of study drug
Supervise application of study drug by unblinded study staff ensuring correct procedure is followed
Monitor participant for 30 minutes after the application of study drug

### Visit 4: Day 2 (Treatment Phase)

On Day 2, the following procedures/assessments will be conducted:
TNSS
Macroscopic nasal examination
Blood collection for study drug level prior to study drug application
Study drug application at site (one dose applied by the participant in the clinic under the supervision of unblinded study staff)

### Visit 5: Day 3 (Treatment Phase)

On Day 3, the following procedures/assessments will be conducted:
TNSS
Macroscopic nasal examination
Study drug application at site (one dose applied by the participant in the clinic under the supervision of unblinded study staff)

### Visit 6: Day 4 (Treatment Phase)

On Day 4, the following procedures/assessments will be conducted:
TNSS
Macroscopic nasal examination
Study drug application at site (one dose applied by the participant in the clinic under the supervision of unblinded study staff)

### Visit 7: Day 5 (Treatment Phase)

On Day 5 (+1 day), the following procedures/assessments will be conducted:
TNSS
Macroscopic nasal examination
Blood collection for study drug level prior to study drug application
Study drug application at site (one dose administration applied by the participant in the clinic under the supervision of study staff)

### Visit 8: Day 8 (Follow-up)

On Day 8 (±1 day), the following procedures/assessments will be conducted:
Physical examination (including body measurements, excluding height)
TNSS
Macroscopic nasal examination
Anterior nares swabs for SA culture (swabs to be retained)

### Visit 9: Day 12 (Follow-up)

On Day 12 (±1 day), the following procedures/assessments will be conducted:
Macroscopic nasal examination
Anterior nares swabs for SA culture (swabs to be retained)

### Visit 10: Day 28 (Follow-up)

On Day 28 (±1 day), the following procedures/assessments will be conducted:
Macroscopic nasal examination
Anterior nares swabs for SA culture (swabs to be retained)

### Demographics

Demographic information to be obtained at screening will include date of birth, gender, ethnicity, and race as described by the participant.

### Adverse Events

Any untoward medical occurrence in the participant's medical condition will be recorded in source and the electronic case report form (eCRF) as an AE, with appropriate follow-up. All AEs occurring during the study (from the date of consent to the end of follow-up [Day 28]), whether or not attributed to the study drug (observed by the Investigator or reported by the participant) will be recorded in source and the eCRF.

### Treatment Score

### Total Nasal Symptom Score

The TNSS will be measured at Baseline (pre-dose on Day 1) and at each study visit until the end of treatment. The TNSS is a subjective measure, and is the sum of 5 individual participant-assessed symptom scores for each of the following symptoms: sneezing, rhinorrhoea, nasal itching, nasal pain and nasal obstruction using ordinal scales with the following grading:

Sneezing, rhinorrhoea, nasal itching, and nasal pain:
0=none, 1=mild, 2=moderate, 3=severe, 4=very severe

Nasal obstruction:
0=breathing through the nose freely and easily. 1=slightly difficult, 2=moderate difficulty, 3=severe difficulty, 4 breathing through nose is very difficult/impossible

Any participant with a grade 3 or 4 nasal tolerability assessment for any of the assessed symptoms should have an additional evaluation by an ENT physician.

### Macroscopic Nasal Examination

Macroscopic nasal examinations will occur at every study visit from Baseline (to the last follow-up visit (Day 28). Nasal examination will be performed by visual inspection of the anterior nasal cavity by the Investigator. The Investigator will be blinded with respect to treatment allocation.

The anterior nares will be examined for mucosal erythema, oedema or irritation and the surrounding nostril examined for crusting, discharge or irritation.

Mucosal erythema or oedema:
0=none, 1=barely perceptible, 2=well-defined, 3=pronounced.

Nasal crusting, discharge or irritation:
0=none, 1=mild, 2=moderate, 3=severe.

Any participant with a grade 2 or 3 for erythema, oedema, nasal crusting, discharge, or irritation should have an additional evaluation by an ENT physician.

### Efficacy Assessments

Efficacy will only be evaluated in participants categorised as persistent carriers of SA. Anterior nares swabs for SA culture will be collected at Screening Visit 1 and 2 (as applicable) and assessed for the presence of SA to determine eligibility. Baseline (Day 1) and follow-up (Day 8, 12 and 28) anterior nares swabs will be collected to determine the change in SA colonisation status from Baseline to Days 12 (primary endpoint), 8 and 28 (secondary endpoint), and to determine recolonisation in participants reporting a negative SA culture at Day 8 (first follow-up) at Days 12 and 28 (subsequent follow-up visits). Colonisation status will be recorded in the source and the eCRF. All anterior nares swabs will be retained until study completion.

### Blood Samples for Study Drug Levels

All participants will have a blood sample taken before dosing at Day 1, Day 2 and Day 5 to measure plasma levels of CBD. Blood samples will be analysed using a validated liquid chromatography-tandem mass spectrometry (LC-MS/MS) method. The limit of detection is 0.2 ng/mL. Haemolysed plasma has an impact on data accuracy and should be avoided during sample collection. Plasma samples may be stored at −20° C. for up to 30 days. However, plasma samples will be shipped to the central laboratory for study drug levels as per the timelines outlined in the Tetra-Q Laboratory Manual. Details on the methods for obtaining and preparing samples for CBD levels are provided in the Tetra-Q Laboratory Manual.

### Anterior Nares Cultures

The clinical study site will collect nasal specimens by swab from the anterior nares of each participant. Specimens will be transferred to medium for culture and identification of SA. All Screening Visit (Visit 1) swabs are to be discarded after the specimens have been transferred to medium for culture. For every subsequent visit, all swabs must be retained. Procedures for collection and processing of swab specimens, and storage of bacterial isolates are found in the Laboratory Manual. Participants must have an anterior nares culture that is positive for SA at Screening Visit 1 to be eligible for Screening Visits 2 and Baseline Visit.

### Bacterial Genotyping and Phenotyping of SA Isolates

In vitro tests will be conducted on all SA isolates collected during the screening, treatment and follow-up phases of the study to determine the minimum inhibitory concentration of CBD and other compounds. For randomised participants, pulsed-field gel electrophoresis will also be conducted to determine strain relatedness between Screening/Baseline SA isolates and isolates recovered during or post-treatment. Additional genetic characterisation of select SA isolates may also be conducted as needed.

### Statistical Considerations

### Statistical and Analytical Plans

All statistical processing will be performed using SAS® version 9.4 or later unless otherwise stated. P-values will be provided for exploratory purposes only.

A statistical analysis plan (SAP), describing all statistical analyses will be provided as a separate document. The SAP will be finalized prior to unblinding of the study treatments.

### Statistical Hypotheses

The purpose of this study is to demonstrate the effectiveness of BTX 1801 presented as 2 different dosage forms to eradicate carriage of SA on Day 12 in the anterior nares of individuals who are persistent carriers of SA. P-values for selected endpoints will be presented to assist in evaluating the outcome of the study. Failure to achieve a statistically significant result does not imply a failed study; results from this study will be used to inform statistical approaches for registration studies.

The primary efficacy point is a negative SA anterior nares culture at Day 12 t in participants who are persistent carriers. The null hypothesis is that there is no difference in the percent of anterior nares cultures that are negative for SA at Day 12 between active BTX 1801 compositions and the combined Vehicle compositions applied twice daily for 5 days to the anterior nares of healthy adults who are nasal carriers of SA.

The alternative hypothesis for this study is that there is a difference in the percent of anterior nares culture that are negative for SA at Day 12 between active BTX 1801 compositions and the combined Vehicle compositions applied twice daily for 5 days.

H₀: Ptrt2=Pveh versus H₁: Ptrt2≠Pveh

Where Ptrt2 and Pveh represent the percentage of negative SA anterior nares culture at Day 12 for the of active BTX 1801 dosing group and combined Vehicle groups respectively.

Should any post-hoc statistical analyses be conducted to present study outcomes, the methods for analysis may be described in the final clinical study report.

### Study Drug Concentration Population

The Study Drug Concentration Population will include all participants who underwent blood sampling for study drug during the study. The Study Drug Concentration Population will be used in all individual and summary presentations of concentration-time data

### Efficacy Population

Participants who complete 5 days of dosing and the follow up visits and provide evaluable culture results will be included in the efficacy population. The Efficacy Population will be used to evaluate the effectiveness of the two different dosage forms of BTX 1801 for the nasal eradication of SA.

### Description of Statistical Methods

All statistical processing will be performed using SAS® version 9.4 or later unless otherwise stated.

Summary statistics will be prepared for the following:
Percentage of persistent carriers with a negative nasal culture for SA on Days 8, 12 and 28.
Percentage of participants who have nasal recolonisation with SA on Day 12 and 28, after a negative nasal culture on Day 8.

The Fisher's Exact test will be used for treatment comparisons of percent eradication of SA in the anterior nares.

Continuous data will be summarised by treatment group using descriptive summary statistics; namely: the number of participants (n), mean, median, standard deviation (SD), minimum value (min), maximum value (max) and 95% confidence interval (CI). The mean will be reported to 1 decimal place more than the level of precision of the data being reported, and the SD will be reported to 2 decimal places more than the level of precision of the data being reported, unless otherwise noted, to a maximum of 4 decimal places.

Summaries at each visit will be calculated using the total number (n) of participants who attended that visit. When summarising change from Baseline, participants are required to have both a non-missing Baseline and non-missing value at the given visit to be summarised.

The analysis for categorical and qualitative data will be summarised using frequencies and percentages. Percentages will be presented to 1 decimal point, unless otherwise specified. The denominators will be the number of participants in each test cohort and for N-value overall.

The mean, standard deviation (SD), median and range will be calculated for the percentage of persistent carriers with a negative nasal culture for SA on Days 8, 12 and 28, and the percentage of participants with nasal recolonisation with SA on Day 12 and/or Day 28 after a negative nasal culture on Day 8. The Fisher's Exact test will be used for treatment comparisons of percent eradication of SA in the anterior nares.

Changes in laboratory parameters from Baseline to Day 8 will be summarised by visit and using shift tables to evaluate for trends. Clinically significant abnormal laboratory findings will be listed.

TNSS and macroscopic nasal examination scores for will be summarised for each visit. In addition, the change from Baseline in the mean scores will be summarised for each visit.

Concomitant medications will be mapped to ATC Level 2 using the World Health Organization (WHO Drug) dictionary. The number and percentage of participants reporting each medication will be summarised. Medications taken by each participant will be listed.

### Analysis of the Study Drug Plasma Levels

Blood levels of study drug will be summarised for Baseline and Days 2 and 5. The mean, SD, median, range, mean coefficient of variation, geometric mean, and coefficient of variation of geometric mean will be presented.

### Baseline Descriptive Statistics

Demographics and Baseline characteristics including age, gender, race, ethnicity, height, and weight, will be summarised overall and by treatment group. Medical history and concomitant medications will be summarised.

### LITERATURE REFERENCES

Appendino G, Gibbons S, Giana A, Pagani A, Grassi G, Stavri M, Smith E, Rahman M M. Antibacterial cannabinoids from Cannabis sativa: a structure-activity study. J Nat Prod. 2008;71(8):1427-30.
Brown A F, Leech J M, Rogers T R, McLoughlin R M. Staphylococcus aureus Colonization: Modulation of Host Immune Response and Impact on Human Vaccine Design. Front Immunol. 2014;4:507.
Burstein, S. Cannabidiol (CBD) and its analogs: a review of their effects on inflammation. Bioorganic & Medicinal Chemistry 23, 1377-1385 (2015).
CTCAE V5.0. Common terminology criteria for adverse events. U.S. Department of Health and Human Services. National Institutes of Health, 27 Nov. 2017. Accessed at: https://ctep.concer.gov/protocolDevelopment/electronic_applications/docs/CTCAE_v5_Quick_Reference_8.5x11.pdf
ICH Harmonised Guideline: Integrated Addendum to ICH E6(R1): Guideline for Good Clinical Practice E6(R2). November 2016.
Ignatowska-Jankowska B, Jankowski M, Glac W, Swiergel AH (2009): Cannabidiol-induced lymphopenia does not involve NKT and NK cells. J Physiol Pharmacol. 60(Suppl 3): 99-103
Agostino J, Ferguson J, Eastwood K and Kirk M The increasing importance of community-acquired methicillin-resistant Staphylococcus aureus infections Med J Aust 2017; 207 (9): 388-393. ∥doi: 10.5694/mja17.00089
Kaplan B, Springs AE, Kaminski NE (2008): The profile of immune modulation by cannabidiol (CBD) involves deregulation of nuclear factor of activated T cells (NFAT). Biochem Pharmacol. 76: 726-737.
Kuehnert M J, Kruszon-Moran D, Hill H A, Prevalence of Staphylococcus aureus Nasal Colonization in the United States, 2001-2002. JID 2006:193
Poovelikunnel T, Gethin G, Humphreys H, Mupirocin resistance: clinical implications and potential alternatives for the eradication of MRSA, Journal of Antimicrobial Chemotherapy, Volume 70, Issue 10, October 2015, Pages 2681-2692, https://doi.org/10.1093/jac/dkv169
Ritchie S R, Isdale E, Priest P, Rainey P B, Thomas M G. The turnover of strains in intermittent and persistent nasal carriers of Staphylococcus aureus. J Infect. 2016;72(3):295-301.

Sakr A, Brégeon F, Mège J L, Rolain J M, Blin O. Staphylococcus aureus Nasal Colonization: An Update on Mechanisms, Epidemiology, Risk Factors, and Subsequent Infections. Front Microbiol. 2018;9:2419.
Sánchez, A. J. & García-Merino, A. Neuroprotective agents: Cannabinoids. Clinical Immunology 142, 57-67 (2012).
van Klingeren, B. & ten Ham, M. J. A. v. L. Antibacterial activity of Δ9-tetrahydrocannabinol and cannabidiol. 42, 9-12 (1976).
van Rijen M, Bonten M, Wenzel R, Kluytmans J. Mupirocin ointment for preventing Staphylococcus aureus infections in nasal carriers. Cochrane Database of Systematic Reviews 2008, Issue 4. Art. No.: CD006216. DOI: 10.1002/14651858.CD006216.pub2.
Whiting, P. F. et al. Cannabinoids for Medical Use: A Systematic Review and Meta-analysis. JAMA 313, 2456-2473 (2015).

1.-21. (canceled)

22. A method for treating or reducing the risk of a bacterial infection, the method comprising topically administering a composition comprising 25 mg-2000 mg of a cannabinoid to a subject in need thereof

23. The method of claim 22, wherein the composition comprises 25 mg-500 mg of a cannabinoid.

24. The method of claim 22, wherein the infection is a topical infection and the composition is administered to the skin a mucosal surface.

25. The method of claim 22, wherein the composition is administered to the eye.

26. The method of claim 22, wherein the infection is an ocular infection and the composition is administered to the eye.

27. The method of claim 22, wherein the composition is administered to nasal mucosal tissue.

28. The method of claim 27, wherein the composition is inhaled.

29. The method of claim 22, wherein the bacterial infection is infection by a gram positive bacteria.

30. The method of claim 29, wherein the gram positive bacteria is: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof

31. The method of claim 22, wherein the bacteria is biofilm-forming bacteria.

32. The method of claim 22, wherein the bacteria is resistant to at least one antibiotic.

33. The method of claim 22, wherein the composition is a ointment or a gel.

34. The method of claim 22, wherein the composition comprises one or more poly (substituted or unsubstituted alkylene) glycols or a derivative thereof

35. The method of claim 22, wherein the composition is gel and comprises at least one of a siloxane and a low molecular weight alcohol and further comprises a viscosity modifier that increase the viscosity of the composition.
